(19) United States
(12) Patent Application Publication
(10) Pub. No.: US 2020/0277648 A1
(43) Pub. Date: Sep. 3, 2020

(54) Recombinant RNA-Dependent RNA Polymerase of RNA Viruses

(71) Applicant: The Governors of the University of Alberta, Edmonton (CA)

(72) Inventors: Matthias Götte, Edmonton (CA); Egor Petrovitch Tchesnokov, Edmonton (CA)

(21) Appl. No.: 16/646,916

(22) PCT Filed: Sep. 4, 2018
(86) PCT No.: PCT/CA2018/051065

§ 371 Date: Mar. 12, 2020

Related U.S. Application Data
(60) Provisional application No. 62/560,553, filed on Sep. 19, 2017.

(51) Int. Cl. C12Q 1/48; C12Q 1/70; C07K 14/005; C12N 5/07; C12N 9/50; A61K 31/7068

(52) U.S. Cl. CPC C12Q 1/48; C12Q 1/70; C07K 14/005; C07K 2319/20; C12N 9/506; A61K 31/7068; C07K 2319/50; C12N 5/0601

(57) ABSTRACT

The present disclosure provides nucleic acids, expression vectors, host cells for producing recombinant viral RNA-dependent RNA polymerase (RdRp) polypeptides of viruses such as Ebola virus. The present disclosure also provides methods and substrates for assaying activity of a RdRp polypeptide or a RdRp complex. Also provided herein are inhibitors of RdRp polypeptides of viruses such as Ebola virus for use in treating or preventing viral infection.

### INCORPORATION BY REFERENCE OF SEQUENCE LISTING

A Sequence Listing is provided herewith as a text file, “UALB-040WO Seq List_ST25.txt” created on Sep. 4, 2018 and having a size of 17 KB. The contents of the text file are incorporated by reference herein in their entirety.

### CROSS-REFERENCE

This application claims the benefit of U.S. Provisional Patent Application No. 62/560,553, filed Sep. 19, 2017, which application is incorporated herein by reference in its entirety.

### INTRODUCTION

Assays, such as, cell-free assays, for identifying drugs that can inhibit replication of viruses are valuable tools for drug discovery and development. However, such assays are challenging to develop as they often require an active viral enzyme, such as, a viral polymerase, as well as identification of an appropriate substrate to test activity of the viral enzyme.

Thus, there is a need for methods for producing active viral polymerases and assays that can measure enzymatic activity and its inhibition.

### SUMMARY

A method for assaying activity of a recombinant RNA-dependent RNA polymerase (RdRp) complex or a recombinant monomeric RdRp of a virus is provided. The method may include incubating a reaction mixture comprising: (i) the recombinant the RdRp complex or the recombinant monomeric RdRp; (ii) a RNA substrate comprising: (a) a primer phosphorylated at the 5′-end and comprising nucleotides, and (b) a template comprising eleven nucleotides, wherein the three nucleotides of primer are complementary to the 3′-end of the template; and (iii) nucleotides; detecting incorporation of at least one nucleotide complementary to the template into the primer, wherein incorporation of at least one nucleotide into the primer indicates that the recombinant RdRp complex or the recombinant monomeric RdRp is active and copies the template in a base-specific manner.

In certain aspects, the recombinant RdRp complex or the monomeric RdRp is from a single-stranded RNA (ss-RNA) virus. In certain aspects, the recombinant RdRp complex is from a negative sense ss-RNA virus (e.g., EBOV, Flu virus, or RSV). In certain aspects, the recombinant monomeric RdRp is from a negative sense ss-RNA virus (e.g., SINV Hanta virus, CCHV, or LASV) or from a positive sense ss-RNA virus (e.g., HCV or Zika virus).

In certain embodiments, the recombinant RdRp complex is from a EBOV, influenza virus, or RSV. In certain aspects, the recombinant monomeric RdRp is from Crimean-Congo hemorrhagic fever (CCHFV), LASV, SINV, HCV or Zika virus.

In certain embodiments, the reaction mixture comprises Mg²⁺.

In certain embodiments, the reaction mixture comprises a candidate agent, wherein lack of complete extension of the primer indicates that the candidate agent is an inhibitor of the RdRp complex or the monomeric RdRp.

In certain cases, detecting incorporation of at least one nucleotide complementary to the template into the primer comprises measuring a fluorescent signal, wherein increase in the fluorescent signal over time indicates incorporation of one or more nucleotides complementary to the template into the primer and decrease in the fluorescent signal over time indicates lack of incorporation of one or more nucleotides complementary to the template into the primer. In certain cases, the period of time is a period of about 400 seconds to 4000 seconds (secs) after all components of the reaction mixture are assembled. In certain cases, the period of time is 1000 secs to 5000 secs after all components of the reaction mixture are assembled. In certain cases, the period of time is 1000 secs to 4000 secs after all components of the reaction mixture are assembled. In certain cases, the fluorescent signal is generated from a dye that intercalates between the template and the extended primer. In certain cases, the dye is dsRNA dye. In certain cases, the dye is a cyanine dye. In certain cases, the cyanine dye is OliGreen, RiboGreen or PicoGreen. In certain cases, the dye is a cyanine dimer dye. In certain cases, the cyanine dimer dye is TOTO®-1 iodide.

Also disclosed herein is a kit comprising (i) a recombinant viral RdRp complex or a recombinant monomeric viral RdRp; (ii) a RNA substrate comprising: (a) a primer phosphorylated at the 5′-end and comprising nucleotides, and (b) a template comprising eleven nucleotides, wherein the three nucleotides of primer are complementary to the 3′-end of the template; and optionally (iii) nucleotides. In certain embodiments, the primer comprises the sequence: ACGC. In certain embodiments, the template comprises the sequence: UUUGUUCGCGU (SEQ ID NO: 39). In certain embodiments, the nucleotides comprise ara-CTP.

In certain embodiments, the viral RdRp complex is a complex expressed by Ebola virus, RSV, or Influenza virus and wherein the viral monomeric RdRp is a monomeric RdRp expressed by zika virus or HCV. The kit may further include a buffer for maintaining a pH suitable for enzymatic activity of RdRp.

The kit may include a divalent metal ion Mg²⁺. In certain embodiments, the viral RdRp complex is a recombinant EBOV RdRp and the divalent metal ion Mg²⁺. In certain embodiments, the viral RdRp complex is a recombinant RSV RdRp and the divalent metal ion Mg²⁺. In certain embodiments, the viral RdRp complex is a recombinant FluB RdRp and the divalent metal ion Mn²⁺.

A method for assaying activity of a recombinant RNA-dependent RNA polymerase (RdRp) complex of Ebola virus (EBOV) is provided. The method may include incubating a reaction mixture comprising: (i) the recombinant EBOV RdRp complex comprising an EBOV L polypeptide and an EBOV VP35 polypeptide; (ii) a RNA substrate comprising: (a) a primer phosphorylated at the 5′-end and comprising at least three nucleotides, and (b) a template comprising at least four nucleotides, wherein the three nucleotides of the primer are complementary to the 3′-end of the template; and (iii) nucleotides; detecting incorporation of at least one nucleotide complementary to the template into the primer, wherein incorporation of the at least one nucleotide into the primer indicates that the recombinant EBOV RdRp is active.

In certain aspects, the recombinant EBOV RdRp further comprises an EBOV VP30 polypeptide. In certain aspects, the primer is at least four nucleotides long. In certain aspects, the template is eleven nucleotides long. In certain aspects, the reaction mixture comprises Mg²⁺.

In certain aspects, the reaction mixture comprises a candidate agent, wherein a lack of incorporation of the at least one nucleotide or an incomplete extension of the primer indicates that the candidate agent is an inhibitor of the EBOV RdRp.

A polyprotein comprising an amino acid sequence for Ebola virus (EBOV) L polypeptide fused via a linker to an amino acid sequence for EBOV VP35 polypeptide, wherein the linker is a cleavable linker is also provided. In certain aspects, the polyprotein further comprises an amino acid for EBOV VP30 polypeptide.

In certain aspects, the cleavable linker comprises a sequence cleaved by a protease.

In certain aspects, the polyprotein further comprises a tag fused to the polyprotein. In certain aspects, the tag comprises a purification tag selected from the group consisting of (Histidine)₆ and streptavidin.

A nucleic acid encoding the polyprotein disclosed herein is also provided. The present disclosure also includes a host cell expressing the polyprotein provided herein. In certain aspects, the host cell comprises the nucleic acid encoding the polyprotein disclosed herein. In certain aspects, the host cell may be an insect cell.

A system for producing a recombinant Ebola virus (EBOV) RNA-dependent RNA polymerase comprising L polypeptide and VP35 polypeptide is also disclosed. Embodiments of the system may include a nucleic acid encoding a polyprotein comprising an amino acid sequence of EBOV L polypeptide and an amino acid sequence of EBOV VP35 polypeptide, wherein the amino acid sequence of the L polypeptide is separated from the amino acid sequence for the VP35 polypeptide by the amino acid for a proteolytic cleavage site; and a nucleic acid encoding a protease that cleaves the cleavage site. In certain aspects, the protease is a tobacco etch virus (TEV) protease. In certain aspects, the polyprotein further comprises an amino acid sequence of EBOV VP30 polypeptide.

Aspects of the present disclosure include a host cell comprising a nucleic acid encoding a polyprotein comprising an amino acid sequence of EBOV L polypeptide and an amino acid sequence of EBOV VP35 polypeptide, wherein the amino acid sequence of the L polypeptide is separated from the amino acid sequence for the VP35 polypeptide by the amino acid sequence for a proteolytic cleavage site; and a nucleic acid encoding a protease that cleaves the cleavage site. In certain aspects, the protease may be a tobacco etch virus (TEV) protease.

In certain embodiments, the polyprotein further comprises an amino acid sequence of EBOV VP30 polypeptide. In certain aspects, host cell is an insect cell. In certain aspects, the host cell includes a vector comprising the nucleic acid encoding the polyprotein.

Also disclosed herein is a kit comprising (i) a recombinant EBOV RdRp comprising an EBOV L polypeptide and an EBOV VP35 polypeptide; (ii) a RNA substrate comprising: (a) a primer phosphorylated at the 5′-end and comprising at least three nucleotides, and (b) a template comprising at least four nucleotides, wherein the three nucleotides of primer are complementary to the 3′-end of the template. In certain aspects, the kit further includes (iii) nucleotides. In certain aspects, the primer comprises the sequence: 5′-ACGC-3′. In certain cases, template comprises the sequence 5′-UUUGUUCGCGU-3′ (SEQ ID NO: 39). In certain aspects, the nucleotides comprise ara-CTP. In certain aspects, the EBOV RdRp further comprises EBOV VP35 polypeptide. In certain aspects, the kit further includes a buffer for maintaining a pH suitable for enzymatic activity of EBOV RdRp. In certain aspects, the kit further includes a divalent metal ion Mg²⁺.

A method of inhibiting activity of a viral RNA dependent RNA polymerase (RdRp) is also disclosed. The method includes contacting the viral RdRp with 2′ 3-hydroxy-cytidine-5′-triphosphate (ara-CTP) or an analog or derivative thereof in an amount effective to inhibit activity of the viral RdRp. In certain embodiments, contacting the viral RdRp comprises administering the ara-CTP or an analog or derivative thereof to a subject having or suspected of having a viral infection. In certain embodiments, the subject has or is suspected of having Ebola virus infection, RSV infection, Influenza virus infection, zika virus infection, and/or HCV infection. In certain embodiments, subject has a viral infection and the administering is for a period of time sufficient to treat the infection. In certain embodiments, the subject is a human.

### BRIEF DESCRIPTION OF THE DRAWINGS

FIG. 1 SDS PAGE migration patterns of purified viral proteins.

FIG. 2 RNA synthesis by recombinant Ebola virus (EBOV) RNA-dependent RNA polymerase (RdRp).

FIGS. 3A and 3B. RNA synthesis by viral RdRp as a function of di-valent metal ions concentration.

FIGS. 4A and 4B. RNA synthesis by viral RdRp as a function of mono-valent metal ion concentration.

FIG. 5. RNA synthesis as a function of primer length.

FIG. 6. RNA synthesis as a function of primer length.

FIG. 7. Chemical structures of cytidine nucleotide substrate analogues.

FIG. 8. RNA synthesis and inhibition.

FIG. 9. RNA synthesis by viral RdRp as a function of incorporation of ara-CMP at position +4 with respect to RNA template.

FIG. 10. RNA synthesis by viral RdRp as a function of incorporation of 2′d-CMP at position +4 with respect to RNA template.

FIG. 11. Analysis of incorporation of ara-CMP and 2′d-CMP during RNA synthesis by viral RdRp.

FIG. 12. Expression of new negative-sense RNA viral RdRp and human mitochondrial RNA polymerase.

FIG. 13. RNA synthesis activity of recombinant purified viral RdRp.

FIG. 14. Development of a high throughput fluorescence-based assay for primed RdRp activity (HTP-FBA-pRdRpa) of a viral recombinant purified RdRp.

FIG. 15. Nucleotide substrate specificity of a high through-put fluorescence-based assay for primed RdRp activity (HTP-FBA-pRdRpa) and inhibition of thereof by a NTP-analogue.

### DETAILED DESCRIPTION

The present disclosure provides methods for producing recombinant RNA-dependent RNA polymerase complexes for ss-RNA viruses such as negative and positive sense RNA viruses. The methods include assays for determining generation of a functional RdRp complex. Systems and kits for practicing the methods are also provided. Also provided herein are assays, substrates, systems, and kits for assaying activity of RdRp complex of RNA viruses such as EBOV, influenza virus (e.g., FluB) or respiratory syncytial virus (RSV) or a monomeric RdRp of Crimean-Congo hemorrhagic fever (CCHFV), LASV, SINV, hepatitis C virus (HCV) or Zika virus. Also disclosed is the use of NTP analogs, such as, ara-CTP, ara-ATP, Ribvirin-TP, and Favipiravir-TP as an inhibitor of RdRp such as, EBOV RdRp complex and its use in a method for treating a viral infection caused by a virus having a RdRp, such as EBOV.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a primer” includes a plurality of primers and reference to “the nucleotides” includes reference to one or more nucleotides, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

As used herein, the term “polyprotein” is meant to refer to a polypeptide which includes amino acid sequences of at least two different proteins, such as, EBOV L polypeptide and EBOV VP35 polypeptide, separated by a cleavage site. The at least two different proteins may be present in any order, e.g., from N-terminus to C-terminus: L polypeptide-cleavage site-VP35 polypeptide or VP35 polypeptide-cleavage site-L polypeptide. The cleavage site present in the polyprotein may be a protease cleavage site. When expressed in a host cell that also expresses a protease that cleaves the cleavage site present between the individual polypeptide sequences in a polyprotein, the polyprotein, upon cleavage yields the individual polypeptides. The individual polypeptides released from cleavage of a polyprotein may be associated with each other via a noncovalent interaction (e.g., electrostatic- or pi-interaction, van der Walls forces, and/or hydrophobic effects) or a covalent bond that is not an amide bond (e.g., a disulphide bond). A polyprotein is generated from a nucleic acid that includes the nucleotide sequences encoding the individual polypeptides operably linked to a single promoter. The nucleotide sequences encoding the individual polypeptides are separated from each other by a nucleotide sequence encoding a proteolytic cleavage site.

The term “about” in relation to a numerical value x means, for example, x±10%.

The word “substantially” does not exclude “completely” e.g. a composition which is “substantially free” from Y may be completely free from Y. The term “substantially” as used herein refers to at least 70%, 80%, 90%, 99%, or 100%. For example, the phrase “substantially pure” in the context of a protein or polypeptide refers to protein or polypeptide preparation that includes less than 30%, 20%, 10%, or lesser contaminating proteins or polypeptides.

The term “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain radioactive or fluorescent labels and may contain not only conventional ribose (e.g., rNTP) and deoxyribose (e.g., dNTP) sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” refers to a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically which can hybridize with naturally occurring nucleic acids in a sequence specific manner analogous to that of two naturally occurring nucleic acids, e.g., can participate in Watson-Crick base pairing interactions. Naturally-occurring nucleotides include uracil, guanine, cytosine, adenine and thymine (U, G, C, A and T, respectively).

The terms “determining”, “measuring”, “evaluating”, “assessing” and “assaying” are used interchangeably herein to refer to any form of measurement and include determining if an element is present or not. These terms include both quantitative and/or qualitative determinations. Assessing may be relative or absolute. “Determining the presence of” includes determining the amount of something present, as well as determining whether it is present or absent.

The terms “ribonucleic acid” and “RNA” as used herein refers to a polymer composed of ribonucleotides.

The term “endogenous” refers to a naturally-occurring biological component of a cell or a virus, i.e., as found in nature.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to a coding sequence.

“Recombinant” as used herein refers to nucleic acid encoding a gene product, or a gene product (e.g., polypeptide) encoded by such a nucleic acid, that has been manipulated by the hand of man, and thus is provided in a context or form in which it is not found in nature. “Recombinant” thus encompasses, for example, a nucleic acid encoding a gene product operably linked to a heterologous promoter (such that the construct that provides for expression of the gene product from an operably linked promoter with which the nucleic acid is not found in nature). For example, a “recombinant L polypeptide” encompasses a L polypeptide encoded by a construct that provides for expression from a promoter heterologous to the L polypeptide coding sequence, L polypeptides that are modified relative to a naturally-occurring L polypeptide (e.g., as in a fusion protein), and the like. It should be noted that a recombinant L polypeptide can have a sequence identical or similar (at least 80%, 85%, 90%, 95%, or 98% identical) to a L polypeptide sequence found in a virus in the nature. Thus, a recombinant polypeptide does not encompass a polypeptide purified from a source where it naturally exists. A recombinant RdRp of a virus refers to a RdRp produced using a nucleic acid encoding the RdRp, where the amino acid sequence of the RdRp is same as or substantially same (at least 80%, 85%, 90%, 95%, or 98% identical) as the amino acid sequence of RdRp of the virus. The nucleic acid may be operably linked to a promoter that controls expression of the polypeptide from the nucleic acid. Recombinant polypeptide does not encompass a polypeptide isolated from a source where it naturally occurs. For example, a recombinant RdRp does not encompass a RdRp isolated from a virus the expresses the RdRp.

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G).

The phrases “operably associated” and “operably linked” refer to functionally related nucleic acid sequences. By way of example, a regulatory sequence is operably linked or operably associated with a protein encoding nucleic acid sequence if the regulatory sequence can exert an effect on the expression of the encoded protein. In another example, a promoter is operably linked or operably associated with a protein encoding nucleic acid sequence if the promoter controls the transcription of the encoded protein. While operably associated or operably linked nucleic acid sequences can be contiguous with the nucleic acid sequence that they control, the phrases “operably associated” and “operably linked” are not meant to be limited to those situations in which the regulatory sequences are contiguous with the nucleic acid sequences they control.

The term “promoter” refers to a nucleic acid sequence that does not code for a protein, and that is operably linked or operably associated to a protein coding sequence such that the transcription of the operably linked or operably associated protein coding nucleic acid sequence is controlled by the promoter. Although typically found 5′ to the protein coding nucleic acid sequence to which they are operably linked or operably associated, promoters can be found in intron sequences as well. Promoters can comprise many elements, including regulatory elements. The term “promoter” is meant to include regulatory sequences operably linked or operably associated with the same protein encoding sequence that is operably linked or operably associated with the promoter. The term “promoter” comprises promoters that are inducible, wherein the transcription of the operably linked nucleic acid sequence encoding the protein is increased in response to an inducing agent. The term “promoter” may also comprise promoters that are constitutive, or not regulated by an inducing agent.

“Nucleotide analogs” include nucleotides having modifications in the chemical structure of the base, sugar and/or phosphate, including, but not limited to sugar-modified ribonucleotides with modifications at the 1′, 2′, 3′ and/or 4′ carbon atoms in which for example the 2′-OH is replaced by a group such as an H, OR, R, halo, SH, SR, NH₂, NHR, NR₂, or CN, wherein R is an alkyl moiety as defined herein. Nucleotide analogs are structurally related to natural nucleoside triphosphates that bind to the active site of RdRps. In contrast to natural nucleotides, nucleotide analogs inhibit RNA synthesis by RdRps.

The term “overhang” refers to a terminal (5′ or 3′) non-base pairing nucleotide resulting from one strand extending beyond the other strand within a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang. The RNA substrates described herein include a template that has a 5′ end that extends beyond the 3′ end of the primer to which it is hybridized. The overhang includes the sequence that is copied as the 3′ end of the primer is extended by activity of a RdRp.

### Methods

The present disclosure provides methods for producing recombinant Ebola virus (EBOV) RNA-dependent RNA polymerase (RdRp) complex comprising EBOV L polypeptide and EBOV VP35 polypeptide. In certain embodiments, the recombinant EBOV RdRp complex may also include EBOV VP30 polypeptide.

The present disclosure also provides methods for assaying for activity of a recombinant EBOV RdRp complex by using a RNA substrate and detection of replication of a template of the RNA substrate. The RNA substrate is also useful for assaying enzymatic activity of RdRp from other RNA viruses, such as, RdRp complex of negative-sense RNA viruses (e.g., influenza virus (Flu), and respiratory syncytial virus (RSV)), monomeric RdRp of negative-sense RNA viruses such as Crimean-Congo hemorrhagic fever (CCHFV), LASV, SINV, and monomeric RdRp of positive-sense RNA viruses such as hepatitis C virus (HCV) and Zika virus. The assays can be used in screening methods for identifying inhibitors of the RdRp.

Various steps and aspects of the methods will now be described in greater detail below.

Production of RdRp Complex

Methods of the present disclosure include recombinantly producing a RdRp complex that includes at least EBOV L polypeptide and EBOV VP35 polypeptide. In some embodiments, the recombinant EBOV RdRp complex may also include EBOV VP30 polypeptide.

In certain aspects, the method may involve producing a host cell that is genetically modified to express a polyprotein comprising EBOV L polypeptide and EBOV VP35 polypeptide, and optionally EBOV VP30 polypeptide. Any suitable host cell can be used for this method. In certain aspects, the host cell may be a eukaryotic cell such as an insect cell. In certain aspects, the insect cell may be an insect cell line capable of recombinant protein production supported by a baculovirus or a bacmid. In certain aspects, the insect cell line may be a Spodoptera frugiperda Sf9 of Sf21 cell line. The host cell may include a nucleic acid encoding the polyprotein comprising an amino acid sequence of EBOV L polypeptide and an amino acid sequence of EBOV VP35 polypeptide, where the amino acid sequence of the L polypeptide is separated from the amino acid sequence for the VP35 polypeptide by the amino acid sequence for a proteolytic cleavage site. The host cell may also include a nucleic acid encoding a protease that cleaves the cleavage site.

In certain aspects, the polyprotein may be a self-cleaving polyprotein which includes the amino acid sequence of the protease as well as the amino acid sequences for the proteolytic cleavage site (cleaved by the protease) interposed between (i) the protease and the EBOV L polypeptide and (ii) the EBOV L polypeptide and EBOV VP35 polypeptide and (iii) the EBOV VP35 polypeptide and the EBOV VP30 polypeptide (if present). The self-cleaving polyprotein may be cleaved by the protease to generate the EBOV L polypeptide and EBOV VP35 polypeptide and the EBOV VP30 polypeptide (if present) which may associate to form RdRp complex. Thus, in certain embodiments, the host cell line may include a nucleic acid that includes a promoter sequence operably linked to the nucleic acid sequence encoding a polyprotein, such as, a self-cleaving polyprotein.

In some embodiments, the methods of the present disclosure include recombinantly producing a RdRp complex that includes at least L polypeptide of Crimean-Congo hemorrhagic fever (CCHFV), LASV, or a Hantavirus such as a SINV. In some case, the recombinantly produced L polypeptide may be conjugated to a heterologous moiety such as a fluorescent molecule, a directly detectable peptide or polypeptide (e.g., green fluorescent protein) or tag such as a peptide tag, e.g., a purification tag, such as, a 6×-His tag, HA tag, etc.

The promoter may be any promoter suitable for expression of the polyprotein encoded by the nucleotide sequence to which the promoter is linked. In certain aspects, the promoter may be a promoter that functions in an insect cell, such as, a Sf9 cell line. The nucleic acid encoding a polyprotein, such as, a self-cleaving polyprotein may be cloned in a vector or a plasmid that includes a single promoter for mediating the expression of the polyprotein. Suitable vectors and plasmids include plasmids and vectors that can support baculovirus mediated protein production and can include bacmids. Exemplary vectors that may be used for production of the polyprotein include pFastBac™ plasmid, pKL-PBac, and the like.

In some embodiments, a baculovirus expression system may be used for producing the RdRp complex. Baculoviruses may be used as expression vectors by inserting genes encoding one or more RdRp polypeptide into the virus genome. The genes are positioned under the control of a very strong baculovirus gene promoter (e.g., polyhedrin) to make a recombinant virus. This promoter drives expression of the RdRp polypeptide gene to make messenger RNA, which in turn makes protein in the recombinant virus-infected cell. The RdRp polypeptide genes or a self-cleaving polyprotein encoding nucleic acid may be inserted into a baculovirus expression vector by using Bac-to-Bac method, as known in the art.

In certain embodiments, at least one polypeptide of the RdRp complex may include a tag that facilitates purification of the RdRp complex. In certain embodiments, the RdRp complex may include two tags, e.g., a first tag fused to a first protein and a second tag fused to a second protein of the RdRp complex to ensure purification of RdRp complexes that includes both the first and second proteins. The first tag and the second tag may be the different. Any suitable tag such as, polyhistidine, avidin, streptavidin, glutathione-S-transferase, FLAG, or biotin may be used.

In some cases, a linker can be interposed between the tag and the RdRp polypeptide and also between a RdRp polypeptide and a protease cleavage site. In addition, a linker may be interposed between the protease and the protease cleavage site. For example, a polyprotein may include the following sequences from the N-terminus to the C-terminus: Protease sequence; linker; protease cleavage site; tag; linker; RdRp polypeptide (e.g., L polypeptide); linker; protease cleavage site; linker; RdRp polypeptide (e.g., VP35 polypeptide); linker; and tag. The linker peptide may have any of a variety of amino acid sequences.

A linker can be a peptide of between about 6 and about 40 amino acids in length, or between about 6 and about 25 amino acids in length. Peptide linkers allowing a degree of flexibility can be used. The linking peptides may have virtually any amino acid sequence, bearing in mind that suitable linkers will have a sequence that results in a generally flexible peptide. Small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly, Ala, or Ser) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers (including, for example, (GS)ₙ, (GS)ₙG, (GSGGS)ₙ (SEQ ID NO: 40) and (GGGS)ₙ (SEQ ID NO: 41), where n is an integer of at least one, e.g., 1-10, 1-2, 3-5, or 2-5, glycine-alanine polymers, alanine-serine polymers, and other flexible linkers known in the art. Glycine and glycine-serine polymers are of interest since both of these amino acids are relatively unstructured, and therefore may serve as a neutral tether between components.

The RdRp complex may be isolated from the host cell expressing the polyprotein by using the one or more tags fused to at least one polypeptide of the RdRp complex. Purification can be achieved using Ni-columns, avidin-beads, or Glutathione-immobilized columns, as examples. In certain embodiments, the isolated RdRp complex may also include other proteins that associate with the complex. For example, the isolated RdRp complex may be associated with one or more heat shock proteins (hsp), such as, hsp70, and/or hsp90 expressed by the host cell.

Any suitable protease may be used for production of the RdRp complex from cleavage of the polyprotein. The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidaselysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase (MMP) cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (SEQ ID NO: 42) (wherein, X represents an arbitrary residue; Hy, a hydrophobic residue), e.g., Pro-X-X-Hy-(Ser/Thr) (SEQ ID NO: 43), e.g., Pro-Leu/Gln-Gly-Met-Thr-Ser (SEQ ID NO: 44) or Pro-Leu/Gln-Gly-Met-Thr (SEQ ID NO: 45). Another example of a protease cleavage site is a plasminogen activator cleavage site, e.g., a uPA or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS (SEQ ID NO: 46), where the protease cleaves between the glutamine and the serine. Another example of a protease cleavage site that can be included in a proteolytically cleavable linker is an enterokinase cleavage site, e.g., DDDDK (SEQ ID NO: 47), where cleavage occurs after the lysine residue. Another example of a protease cleavage site that can be included in a proteolytically cleavable linker is a thrombin cleavage site, e.g., LVPR. Additional suitable linkers comprising protease cleavage sites include linkers comprising one or more of the following amino acid sequences: LEVLFQGP (SEQ ID NO: 48), cleaved by PreScission protease; a thrombin cleavage site, e.g., CGLVPAGSGP (SEQ ID NO: 49); SLLKSRMVPNFN (SEQ ID NO: 50) or SLLIARRMPNFN (SEQ ID NO: 51), cleaved by cathepsin B; SKLVQASASGVN (SEQ ID NO: 52) or SSYLKASDAPDN (SEQ ID NO: 53), cleaved by an Epstein-Barr virus protease; RPKPQQFFGLMN (SEQ ID NO: 54) cleaved by MMP-3 (stromelysin); SLRPLALWRSFN (SEQ ID NO: 55) cleaved by MMP-7 (matrilysin); SPQGIAGQRNFN (SEQ ID NO: 56) cleaved by MMP-9; DVDERDVRGFASFL (SEQ ID NO: 57) cleaved by a thermolysin-like MMP; SLPLGLWAPNFN (SEQ ID NO: 58) cleaved by matrix metalloproteinase 2(MMP-2); SLLIFRSWANFN (SEQ ID NO: 59) cleaved by cathespin L; SGVVIATVIVIT (SEQ ID NO: 60) cleaved by cathepsin D; SLGPQGIWGQFN (SEQ ID NO: 61) cleaved by matrix metalloproteinase 1(MMP-1); KKSPGRVVGGSV (SEQ ID NO: 62) by urokinase-type plasminogen activator; PQGLLGAPGILG (SEQ ID NO: 63) cleaved by membrane type 1 matrixmetalloproteinase (MT-MMP); HGPEGLRVGFYESDVMGRGHARLVHVEEPHT (SEQ ID NO: 64) cleaved by stromelysin 3 (or MMP-11), thermolysin, fibroblast collagenase and stromelysin-1; GPQGLAGQRGIV (SEQ ID NO: 65) (cleaved by matrix metalloproteinase 13 (collagenase-3); GGSGQRGRKALE (SEQ ID NO: 66) cleaved by tissue-type plasminogen activator (tPA); SLSALLSSDIFN (SEQ ID NO: 67) cleaved by human prostate-specific antigen; SLPRFKIIGGFN (SEQ ID NO: 68) cleaved by kallikrein (hK3); SLLGIAVPGNFN (SEQ ID NO: 69) cleaved by neutrophil elastase; and FFKNIVTPRTPP (SEQ ID NO: 70) cleaved by calpain (calcium activated neutral protease). In certain aspects, the protease may be tobacco etch virus (TEV) protease, furin, chymotrypsin, or caspase. In certain aspects, the TEV protease may be a native TEV or a mutated TEV, such as, AcTEV™ protease.

The activity of RdRp complex such as a recombinant EBOV RdRp complex may be assayed using the methods described herein. In certain embodiments, the method for assaying activity of a recombinant RNA-dependent RNA polymerase (RdRp) complex may include incubating a reaction mixture comprising (i) the recombinant RdRp complex (of EBOV, RSV, or Flu) or a monomeric RdRp (HCV or Zika virus); (ii) a RNA substrate comprising: (a) a primer phosphorylated at the 5′-end and comprising at least three nucleotides, and (b) a template comprising at least four nucleotides, where the three nucleotides of the primer are complementary to the 3′-end of the template; and (iii) nucleotides; detecting incorporation of at least one nucleotide complementary to the template into the primer, where incorporation of the at least one nucleotide complementary to the template into the primer indicates that the recombinant RdRp is active and copies the template in a base-specific manner.

In certain aspects, the recombinant RdRp complex may be any recombinant RdRp complex described herein. In certain embodiments, the recombinant RdRp complex may an EBOV RdRp complex comprising an EBOV L polypeptide and an EBOV VP35 polypeptide and optionally, an EBOV VP30 polypeptide. In certain embodiments, the recombinant RdRp complex may be a RdRp of RSV or Flu virus, e.g., FluA virus or FluB virus. In certain embodiments, the RdRp is a monomeric RdRp from Crimean-Congo hemorrhagic fever (CCHFV), LASV, SINV, HCV or Zika virus. In certain aspects, the monomeric RdRp may be a non-structural protein (NS5) RdRp of Zika virus generated as disclosed herein, for example. In certain aspects, the monomeric RdRp may be L polypeptide of CCHFV, LASV, or SINV. In certain embodiments, the recombinant RdRp complex may include recombinant P polypeptide and recombinant L polypeptide of RSV. In certain embodiments, the recombinant RdRp complex may include recombinant PA polypeptide, recombinant PB1 polypeptide, and recombinant PB2 polypeptide of FluB virus. In certain aspects, the RdRp complex of RSV and FluB virus may have been generated using the methods described herein. For example, the individual polypeptides of the complex may have been expressed as a self-cleaving polyprotein.

In some embodiments, the assay may be performed on human mitochondrial RNA polymerase and/or human RNA polymerase II. In some embodiments, an inhibitor of a viral RdRp identified using the methods, compositions, kits, or systems disclosed herein may be assayed using human mitochondrial RNA polymerase and/or human RNA polymerase II to determine whether the inhibitor may have an off-target effect.

The RNA substrate may be partially double stranded structure formed by base pairing between nucleotides of the primer that are complementary to the 3′-end of the template. The remaining nucleotides in the template form an overhang that is copied by an active RdRp and incorporated into the primer thereby extending the primer. In certain embodiments, the RNA substrate may include a 5′ phosphorylated primer that is a 4-mer and a template that is a 5-mer, a 6-mer, a 7-mer, a 8-mer, a 9-mer, 10-mer, or a 11-mer and may include 3 or 4 nucleotides at its 3′-end that are complementary to the primer. Incorporation of a nucleotide that is complementary to the template into the primer may be detected by using rNTP that are detectably labeled. For example, the rNTP may be radioactively labeled. In certain aspects, extension of the primer in a base-specific manner by incorporation of nucleotides complementary to the sequence of the template may be detected by using a combination of detectably labeled nucleotide(s) and sequencing or length based separation of the extended primer or incorporation of a dye that intercalates between double stranded nucleic acid (RNA:RNA; RNA:DNA; or DNA:DNA). In certain aspects, the primer may be a 3-mer having the sequence 5′-ACG-3′ or a 4-mer having the sequence 5′-ACGC-3′ (SEQ ID NO: 71) and the template may be at least 6 nucleotides long and may include 3 or 4 nucleotides at its 3′-end that are complementary to the primer. In other embodiments, the template may be a 11-mer or longer. In other embodiments, the template may be up to 11 nucleotides long. In some embodiments, the template may comprise the sequence 5′-UUUGUUCGCGU-3′ (SEQ ID NO:30) or 5′-UUUGUUCGCGU-3′ (SEQ ID NO: 39). In some embodiments, the template may comprise the sequence 5′-UGCGCUUUUUU-3′ (SEQ ID NO: 72). In some embodiments, the template may comprise the sequence 5′-UGCGAUCUUUU-3′ (SEQ ID NO: 73). In other embodiments, the template may be up to 18 nucleotides long. In some embodiments, the template may comprise the sequence 5′-UGCGCUUUUUUACCCCCC-3′ (SEQ ID NO: 74). The NTPs present in the reaction mixture may include ATP, GTP, and CTP.

In certain aspects, the reaction mixture may include a source of a catalytic metal ion, such as, a monovalent or a divalent metal ion. In certain aspects, the reaction mixture may include a source of Mg²⁺ ions. The reaction mixture may also include additional components required for RdRp-mediated incorporation of nucleotides into the primer of the RNA substrate in a template dependent manner. In certain aspects, the reaction mixture may include a buffer (e.g., a Tris or phosphate buffered saline, or HEPES, or another buffer to maintain a physiological pH, e.g., pH 7-7.5, for example, pH 7.4), salt (e.g., NaCl or KCl), disulphide reducing agent (e.g., DTT, -mercaptoethanol, or TCEP (tris(2-carboxyethyl)phosphine), NTPs, RNA substrate, a source of divalent metal ion (e.g., a metal halide, such as, MgCl₂ or MnCl₂), and the RdRp complex or a monomeric RdRp.

The concentration of the constituent reagents in the reaction mixture can be determined empirically. In some instances, a range of concertation of the reagents may be effective in providing the conditions sufficient for RdRp activity. For example, salt (e.g., NaCl) concentration may range from 0 mM-200 mM, e.g., 10 mM-100 mM, e.g., 20 mM; buffer concentration may range from 10 mM-200 mM, e.g., 10 mM-100 mM or 20 mM-50 mM, e.g., 25 mM; the RNA substrate may include the template and primer at a ratio of 1:200 and the concentration of the primer may be in the range of 100 μM-500 μM, e.g., 150 μM-300 μM; NTPs may be included at a concentration of 0.1 μM-200 μM, e.g., 1 μM-150 μM or μM-150 μM; source of divalent metal ion (e.g., MnCl₂ or MgCl₂) may be included at a concentration of 1 mM-25 mM, e.g., 2 mM-10 mM; the RdRp complex (e.g., RdRp complex of FluB, RSV, or EBOV) or a monomeric RdRp (e.g., Crimean-Congo hemorrhagic fever (CCHFV), LASV, SINV, HCV or Zika virus RdRp) may be included at a concentration of 0.1-1 μM.

In preparing the reaction mixture, the various constituent components can be added in any convenient order. For example, the various constituent components (other than RdRp) can be assembled in a single mixture and the assay initiated by adding the RdRp. In a certain example, the various constituent components (other than a source of a divalent metal ion) can be assembled in a single mixture and the assay initiated by adding the source of a divalent ion (e.g., Mg²⁺ or Mn²⁺) to the mixture.

In certain aspects, the incubating may be carried out under conditions suitable of RdRp activity and may include incubating at room temperature, 30° C., or at 37° C. The length of incubation may vary and may range from 5 min-10 hrs, such as 15 min-5 hrs, 30 min-2 hrs, 1 hr-2 hrs.

Aliquots of from the reaction mixture may be obtained at various time points and the polymerase activity terminated by any effective means, such as, by heat denaturation, precipitation (e.g., isopropanol or ethanol mediated precipitation of protein), and/or a quenching, e.g., adding EDTA and/or formamide. The aliquots may be assessed to determine presence of extension products of the primer generated by replication of the template.

Activity of RdRp may be measured by detecting incorporation of at least one nucleotide complementary to the template sequence into the primer. For example, the incorporated nucleotide may be a detectably labeled nucleotide that is complementary to the first nucleotide in the overhang region of the template. The incorporation of the labeled nucleotide at the first position (+1) in the primer extension product facilitates not only detection of incorporation of the first nucleotide but also visualization of longer primer extension products, e.g., those separated based on size on a denaturing gel. In some embodiments, the separated primer extension products may be sequenced to determine whether the nucleotides incorporated into the primer are complementary to the sequence of the template. In some embodiments, incorporation of one or more nucleotides into the primer may be measured by detecting a signal generated by incorporation of a dye that intercalates in double stranded nucleic acid. In some embodiments, the signal generated by incorporation of a dye that intercalates in double stranded nucleic acid may be measured over time to follow the extension of the primer and/or accumulation of extension products from additional primer extension over time (e.g., as more primers are extended by the RdRp).

In certain examples, the reaction products generated in the reaction mixture may be analyzed by separating the reaction products in a denaturing gel that resolves the reaction products on the basis of size and determining the size of the separated reaction products. In some instances, a complete replication of the template in the RNA substrate indicates that the RdRp complex is active. In some instances, the reaction mixture may include a detectably labeled rNTP that is incorporated at a specific location in the extended primer and where incorporation of the detectably labeled rNTP is indicative of activity of the RdRp complex. In some instances, formation of a reaction product having a length and sequence indicative of complete replication of the template is indicative of activity of the RdRp complex. In some instances, extension of the primer may be assayed by increase in fluorescent signal of dye that intercalates between the template and the primer as the primer is extended. The fluorescent signal is proportional to the length of the ds nucleic acid. An increase in signal over time is indicative of elongation of the primer.

Screening Methods

Also provided herein are methods for screening for agents that inhibit activity of a RdRp complex (e.g., a recombinant RdRp complex of EBOV, influenza virus, or RSV) or a monomeric RdRp (e.g. a recombinant monomeric RdRp of Crimean-Congo hemorrhagic fever (CCHFV), LASV, SINV, Zika virus, or HCV). The method may include assaying the activity of a RdRp as described herein, in the presence of a candidate agent wherein inhibition of extension of the 5′phosphorylated primer in a template dependent fashion identifies the candidate agent as an inhibitor of the RdRp activity. In certain aspects, the methods for screening for agents that inhibit activity of a RdRp complex or a monomeric RdRp may be a high-throughput method in which at least 10, 30, 300, 1000, 3000, 10,000, 30,000, 100,000, 300,000, 1000,000, or 3 million candidate agents are tested. The high-throughput screening may facilitate rapid identification of agents that inhibit activity of a RdRp. Such rapid identification can be useful in scenarios such as testing for inhibitors in the event of an outbreak of a viral infection. Such screening methods are also useful for testing a large number of candidate agents thereby increasing the chances of identifying an inhibitor.

Candidate agents of interest for screening include biologically active agents of numerous chemical classes, primarily organic molecules, although including in some instances, inorganic molecules, organometallic molecules, immunoglobulins, genetic sequences, etc. Also of interest are small organic molecules, which comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A plurality of assays may be run in parallel with different concentrations of a candidate agent to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in polymerase activity.

As noted herein 2′ 3-hydroxy-cytidine-5′-triphosphate (ara-CTP), which is the triphosphate form of the approved anti-cancer drug cytarabine, is an inhibitor of EBOV RdRp complex. Thus, in certain cases, ara-CTP may be included in the screening assay as a positive control. In addition, ara-CTP may be used as a benchmark for comparing efficacy of additional agents in treatment of infection caused by viruses disclosed herein.

In certain cases, the candidate agents may include nucleoside analogues, such as, non-obligate chain terminators similar to ara-CTP or ara-ATP. In certain cases, the candidate agents may be 2′-C-methyl-nucleoside, e.g., 2′-C-methyl-cytidine or 2′-O-methyl-nucleoside. The assay may involve determining lack of extension of the primer sequence or incomplete extension of the primer, where a lack of extension of the primer sequence or incomplete extension of the primer identifies the candidate agent as an inhibitor of the RdRp.

The assaying may comprise contacting the candidate agent to a reaction mix that includes a RdRp such as a recombinant EBOV RdRp complex or a recombinant monomeric RdRp complex of a virus as described herein, a RNA substrate, rNTPs, a source of divalent metal ion; measuring the activity of the RdRp in the reaction mix, comparing the measured activity to the activity of a control reaction mix that includes recombinant EBOV RdRp complex, a RNA substrate, rNTPs, a source of divalent metal ion but not the candidate agent being tested; and identifying a candidate agent that decreases the RdRp activity.

In certain aspects, a decrease in the RdRp activity is indicated when the amount of replication product generated from extension of the primer of the RNA substrate is decreased as compared to the amount produced in absence of the candidate agent. The replication product may be an extension product that includes an addition of a one nucleotide, two nucleotides, three nucleotides, four nucleotides, five nucleotides, six nucleotides, seven nucleotides to a 5′phosphorylated 3-mer or 4-mer primer by replication of the sequence of the template annealed to the primer, where the template is a 4-mer or longer, such as, a 11-mer.

In certain aspects, a decrease in the RdRp activity is indicated when a replication product generated from extension of the primer of the RNA substrate is shorter than the replication product generated in absence of the candidate agent.

In certain aspects, a decrease in the RdRp activity is indicated when there is a decrease in the amount of replication product generated from extension of the primer of the RNA substrate as compared to the amount generated in absence of the candidate agent. A decrease as well as absence of formation of a replication product may be determined by determining a decrease in or absence of signal generated by incorporation of a labeled nucleotide into a replication product. In certain cases, the label may be a radioactive label or a fluorescent label. In certain cases, the signal being measured is the signal generated from incorporation of a dye in the double stranded product generated upon extension of a primer, as described herein. Decrease or absence of a signal from the dye incorporated into double stranded RNA or RNA/DNA hybrid indicated that the candidate agent is an inhibitor of the RdRp used in the assay.

In certain embodiments, the screen may be performed in a high throughput format, such as, in multi-well plates for screening a plurality of candidate agents, e.g., 10, 1000, 10,000, 100,000 or more agents in parallel.

Agents identified as inhibitors of RdRp activity may be further characterized using cell-based assays or animal models to test their activity as inhibitors of replication of the virus.

### Compositions

The present disclosure also provides nucleic acids encoding the RdRp polypeptide(s), RdRp polypeptides and RdRp complexes comprising RdRp polypeptides, cells expressing RdRp polypeptide(s), and RNA substrates for assaying RNA polymerization activity of RdRp polypeptides.

In some cases, a nucleic acid of the present disclosure comprises, in order from 5′ to 3′ and in operable linkage: a) a promoter that is functional in a eukaryotic cell, such as, an insect cell; b) a nucleotide sequence encoding a polyprotein comprising, in order from N-terminus to C-terminus: i) an EBOV L polypeptide; ii) a proteolytically cleavable linker; and iii) an EBOV VP35 polypeptide. In some cases, the polyprotein comprises a protease that cleaves the proteolytically cleavable linker. Thus, for example, in some cases, a nucleic acid of the present disclosure comprises, in order from 5′ to 3′ and in operable linkage: a) a promoter that is functional in a eukaryotic cell; b) a nucleotide sequence encoding a polyprotein comprising, in order from N-terminus to C-terminus: i) protease; ii) a proteolytically cleavable linker cleavable by the protease; (iii) an EBOV L polypeptide; iv) the proteolytically cleavable linker; and v) an EBOV VP35 polypeptide. In some cases, a nucleic acid of the present disclosure comprises, one or more tag encoding sequence and thus comprises in order from 5′ to 3′ and in operable linkage: a) a promoter that is functional in a eukaryotic cell; b) a nucleotide sequence encoding a polyprotein comprising, in order from N-terminus to C-terminus: i) protease; ii) a proteolytically cleavable linker cleavable by the protease; (iii) a tag; (iv) an EBOV L polypeptide; v) the proteolytically cleavable linker; vi) an EBOV VP35 polypeptide; and vii) a tag which may be different from the (iii) tag.

Suitable promoters include, e.g., a polyhedron promoter, a CMV promoter, an SV40 promoter, and the like. In some cases, the nucleic acid is inserted into an expression vector to generate a recombinant expression vector. Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, human immunodeficiency virus (HIV)-based lentivirus vectors, murine leukemia virus (MVL)-based gamma retrovirus vectors, and the like), P1-based artificial chromosomes, yeast plasmids, yeast artificial chromosomes, and any other vectors specific for specific hosts of interest (such as E. coli, mammalian cells, insect cells, or yeast cells).

The present disclosure provides genetically modified host cells, where the genetically modified host cells are genetically modified with a nucleic acid(s) or recombinant expression vector(s) of the present disclosure.

Suitable host cells include eukaryotic cells, such as yeast cells, insect cells, and mammalian cells. In some cases, the host cell is a cell of an insect cell line. In some cases, the host cell is a cell of a mammalian cell line. Suitable mammalian cell lines include human cell lines, non-human primate cell lines, rodent (e.g., mouse, rat) cell lines, and the like.

Methods for introduction of nucleic acids into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated.

Also provided herein is a RdRp complex comprising EBOV L polypeptide and EBOV VP35 polypeptide where at least one of the polypeptides is a fusion protein comprising a tag sequence, where the tag sequence is a purification tag, such as, polyhistidine or GST.

### Systems

The present disclosure also provides systems which find use, e.g., in practicing the subject methods. The present disclosure provides nucleic acids comprising nucleotide sequences encoding a RdRp polypeptide of the present disclosure, and recombinant expression vectors comprising the nucleic acids. Thus, the present disclosure provides recombinant expression vectors comprising nucleotide sequences encoding RdRp polypeptide of the present disclosure. The present disclosure provides host cells genetically modified with a nucleic acid or a recombinant expression vector of the present disclosure.

Systems of the present disclosure may include a nucleic acid encoding a polyprotein comprising an amino acid sequence of EBOV L polypeptide and an amino acid sequence of EBOV VP35 polypeptide, wherein the amino acid sequence of the L polypeptide is separated from the amino acid sequence for the VP35 polypeptide by the amino acid for a proteolytic cleavage site and a nucleic acid encoding a protease that cleaves the cleavage site.

In certain aspects, the system may include a plasmid or a vector comprising a nucleic acid encoding a self-cleaving polyprotein comprising an amino acid sequence of a protease; amino acid sequence of a cleavage site for the protease; an optional tag sequence; an amino acid sequence of EBOV L polypeptide; amino acid sequence of a cleavage site for the protease; an amino acid sequence of EBOV VP35 polypeptide; an optional tag sequence; optionally amino acid sequence of a cleavage site for the protease; and optionally amino acid sequence for the EBOV VP30 polypeptide. A single promoter that is functional in a eukaryotic cell may control the expression of the polyprotein. The amino acid sequence of a protease may be present at the N-terminus or the C-terminus of the polyprotein. The EBOV RdRp complex polypeptides may be present in any order in the polyprotein. In some cases, the polyprotein comprises the following order of the individual polypeptides from the N-terminus to the C-terminus: amino acid sequence of a protease; amino acid sequence of a cleavage site for the protease; tag sequence; an amino acid sequence of EBOV L polypeptide; amino acid sequence of a cleavage site for the protease; an amino acid sequence of EBOV VP35 polypeptide.

The large L, viral protein VP35 and VP30 polypeptides may have the amino acid sequence of a EBOV L, VP35, and VP30 polypeptides expressed by EBOV in nature. In certain cases, the amino acid sequences of the L, VP35 and VP30 (if present) are the amino acid sequences of L, VP35 and VP30 polypeptides, respectively, of Zaire ebolavirus (ZEBOV), Sudan ebolavirus (SEBOV), Reston ebolavirus (REBOV), Ivory Coast ebolavirus (CIEBOV), or Bundibugyo ebolavirus species.

In certain cases, the EBOV L polypeptide may have the amino acid sequence as set out in GenBank Accession No.: AKG65102.1, UniProt Accession No.: Q05318.2, Q6V1Q2.1, Q8JPX5.1, Q91DD4.1, or Q5XX01.1.

In certain cases, the EBOV VP35 polypeptide may have the amino acid sequence as set out in GenBank Accession No.: AKG65095; AAD14582.1; SCD11532.1; AKL91131.1; AKL91122.1; AKL91104.1; ABY75322.1; or ALT19750.1.

In certain cases, the EBOV VP30 polypeptide may have the amino acid sequence as set out in GenBank Accession No.: AKG65100; SCD11537.1; AKL91126.1; AKU75585.1; or AAD14587.1.

In certain cases, the Sin Nombre RdRp may have a sequence as set out in GenBank Accession No.: AIA08878 or AIA08875.

In certain cases, the Crimean-Congo hemorrhagic fever virus RdRp may have a sequence as set out in GenBank Accession No.: AXH37997, AXH37996, or AXH37995.

In certain cases, the Lassa virus RdRp may have a sequence as set out in GenBank Accession No.: 5J1P_A, 5J1N_A, 5IZH_A, or 4MIW_A.

The system may include host cells that include nucleic acids or expression vectors as disclosed herein.

### Kits

Also provided by the present disclosure are kits. The kits include one or more reagents useful in practicing the methods of the present disclosure. In certain aspects, the kits include (i) a recombinant EBOV RdRp comprising an EBOV L polypeptide and an EBOV VP35 polypeptide; (ii) a RNA substrate comprising: (a) a primer phosphorylated at the 5′-end and comprising at least three nucleotides, and (b) a template comprising at least six nucleotides, wherein the three nucleotides of primer are complementary to the 3′-end of the template. In certain aspects, the kits also include ribonucleotides (rNTPs). In certain cases, at least one of the rNTPs may be detectably labeled, e.g., labeled with a radioactive label or a heavy atom or a fluorescent label.

In certain embodiments, the RNA substrate may include a 5′ phosphorylated primer that is a 4-mer and a template that is a 7-mer, a 8-mer, a 9-mer, 10-mer, or a 11-mer and may include 4 nucleotides at its 3′-end that are complementary to the primer. In certain aspects, the primer may be a 3-mer having the sequence 5′-ACG-3′ or a 4-mer having the sequence 5′-ACGC-3′ (SEQ ID NO: 71) and the template may be at least 6 nucleotides long and may include 3 or 4 nucleotides at its 3′-end that are complementary to the primer. In other embodiments, the template may be a 11-mer or longer. In other embodiments, the template may be up to 11 nucleotides long. In some embodiments, the template may comprise the sequence 5′-UUUGUUCGCGU-3′ (SEQ ID NO: 39) and the rNTPs supplied in the kit may comprise ATP, GTP, and CTP.

In certain aspects, the kit may also include ara-CTP for use as a positive control in assays for inhibitors of EBOV RdRp complex.

In certain aspects, the kit may include a buffer, a source of divalent metal ions (e.g., Mg²⁺ or Mn²⁺), salt, etc.

The methods, substrates, systems and kits of the present disclosure find use in a variety of different applications, including isolating functional RdRp complexes, measuring activity of isolated RdRp complexes, and identifying agents that inhibit activity of RdRp and are potential inhibitors of viral replication and may be useful in treating viral infections, such as, EBOV infection.

### Methods for Inhibiting Viral RdRp

The present disclosure also provides methods for inhibiting viral RdRp. Such methods are useful in treating viral infections. The term “treating” or “treatment” of a viral infection includes providing a clinical benefit to a subject, and includes: (1) inhibiting the viral replication, i.e., arresting or reducing the development of the infection or its symptoms, or (2) relieving the infection, i.e., causing regression of the infection or its clinical symptoms. In certain embodiments, the inhibition of viral RdRp results in inhibition of replication of the virus and a decrease in viral titers.

In certain methods, a method for inhibiting RdRp may include contacting the viral RdRp with an inhibitor of the RdRp in an amount effective to inhibit the viral RdRp. In certain embodiments, the contacting may include administering the inhibitor to a subject suspected of or diagnosed as having a viral infection. In certain embodiments, the subject may be suspected of or may be diagnosed as having an EBOV infection, RSV infection, HCV infection, Influenza infection, Crimean-Congo hemorrhagic fever (CCHFV) infection, LASV infection, SINV infection, or Zika virus infection.

In certain cases, the method of inhibiting a viral infection may include contacting the viral RdRp with 2′β-hydroxy-nucleoside-5′-triphosphate or an analog or derivative thereof in an amount effective to inhibit activity of the viral RdRp. In certain embodiments, contacting a viral RdRp with an inhibitor of viral RdRp, such as, ara-nucleotide or an analog or derivative thereof may include administering the RdRp inhibitor to a subject having or suspected of having a viral infection.

In certain cases, the method of inhibiting a viral infection may include contacting the viral RdRp with 2′J3-hydroxy-cytidine-5′-triphosphate (ara-CTP) or an analog or derivative thereof in an amount effective to inhibit activity of the viral RdRp. In certain embodiments, contacting a viral RdRp with an inhibitor of viral RdRp, such as, ara-CTP or an analog or derivative thereof may include administering the RdRp inhibitor to a subject having or suspected of having a viral infection.

In certain aspects, a method of method of inhibiting a viral infection may include contacting the viral RdRp with Arauridine-5′-Triphosphate (Ara-UTP) or an analog or derivative thereof in an amount effective to inhibit activity of the viral RdRp (e.g., EBOV RdRp complex). In certain embodiments, contacting a viral RdRp with an inhibitor of viral RdRp, such as, ara-UTP or an analog or derivative thereof may include administering the RdRp inhibitor to a subject having or suspected of having a viral infection.

In certain aspects, a method of method of inhibiting a viral infection may include contacting the viral RdRp (e.g., EBOV RdRp complex) with (−)-L-2′, 3′-dideoxy-3′-thiacytidine (Lamivudine, commonly called 3TC).

In certain aspects, a method of method of inhibiting a viral infection may include contacting the viral RdRp (e.g., EBOV RdRp complex) with (−)-(2S,4S)-1-(2 hydroxymethyl-1,3-dioXolan-4-yl) cytosine.

In certain aspects, a method of method of inhibiting a viral infection may include contacting the viral RdRp (e.g., EBOV RdRp complex) with Zidovudine (ZDV), also known as azidothymidine (AZT).

In certain aspects, a method of method of inhibiting a viral infection may include contacting the viral RdRp (e.g., EBOV RdRp complex) with non-obligate chain terminators such as, Sofosbuvir.

Any route of administration may be used for treating a viral infection, including, but not limited to local, such as delivery into the effected tissue, oral, catheter mediated, intrathecal, buccal, parenteral, intraperitoneal, intradermal, transdermal, etc., administration.

A RdRp may be formulated into a pharmaceutical composition. Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, non-immunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant. The RdRp inhibitors can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population).

The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lies within a range of circulating concentrations that include the ED₅₀ with low toxicity. The dosage can vary within this range depending upon the dosage form employed and the route of administration utilized.

In certain embodiments, the viral RdRp inhibitors disclosed herein may be used to prevent a viral infection. For example, the viral RdRp inhibitors disclosed herein may be administered prophylactically to subjects susceptible to viral infection, e.g., newborns, infants, children, the elderly, or a person having a compromised immune system. In certain cases, the viral RdRp inhibitors disclosed herein may be administered to people living in proximity to or coming in contact with persons having a viral infection or suspected of having a viral infection, such as, Ebola virus infection, RSV infection, Influenza virus infection, zika virus infection, and/or HCV infection.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Methods

Protein Expression, Purification and RNA Synthesis Assays.

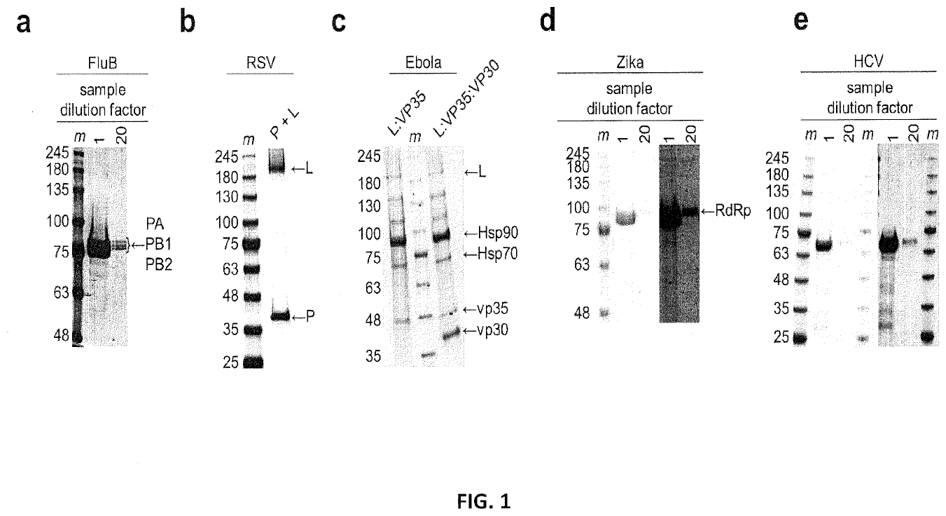

All virus and viral protein specific information as well as pertinent details of protein expression, purification and RNA synthesis assays are summarized in Table 1. HCV RdRp was produced in Escherichia coli as previously described (Ferrari, E. et al. J Virol 73, 1649-1654 (1999)). The pFastBac-1 (Invitrogen) plasmid with the codon-optimized synthetic DNA sequences coding for viral proteins or protein complexes (GenScript) was used as a starting material for viral protein expression in insect cells (Sf9, Invitrogen). We employed the MultiBac (Geneva Biotech) system according to protocols provided by Drs. Garzoni, Bieniossek and Berger (Berger, I., et al. Nature biotechnology 22, 1583-1587 (2004); Bieniossek, C., et al. Current protocols in protein science/editorial board, John E. Coligan, et al. Chapter 5, Unit 5 20 (2008)). Viral proteins and protein complexes were purified using the His- or Strep (Table 3) tag-affinity chromatography according to the manufacturer's specifications (IBA and ThermoScientific, respectively). The identity of the purified viral proteins and protein complexes was confirmed by mass spectrometry analysis (Dr. Jack Moore, Alberta Proteomics and Mass Spectrometry). The presence of insect heat shock proteins precluded reasonable estimation of the protein complex concentration in the Ebola RdRp samples. The RdRp complexes of FluB and RSV did not show significant presence of insect cell proteins (FIG. 1). Therefore, their protein concentration as well as the concentration of Zika and HCV RdRp was determined based on calculated extinction coefficients (GPMAW-lite free software, Alphalyse). Amicon-Ultra (Millipore) centrifugal membranes were used to concentrate purified protein samples.

Data Acquisition, Quantification and Analysis.

The reactions conditions were chosen so that the RNA synthesis product formation was linear with respect to time. Thus, a 30-minute time point was chosen to stop all the reactions involving titration of nucleotide substrates. Reaction substrates and products were resolved through denaturing 8 M urea 15 or 20% PAGE, visualized and quantified by phosphorimaging. Incorporated nucleotide product fraction was plotted versus nucleotide substrate concentrations and fitted to Michaelis-Menten equation using Prism software (GrapPad).

### Example 1: Expression of EBOV RdRp

Negative-sense RNA viruses such as influenza viruses, Measles virus, Mumps virus, respiratory syncytial virus (RSV), and Ebola virus (EBOV) are important human pathogens. Unfortunately, effective antiviral treatments are often not available (Griffiths, C., et al. Clinical microbiology reviews 30, 277-319 (2017); Feams, R. & Plemper, R. K. Virus research 234, 87-102 (2017); Martin, B. et. al. Antiviral research (2017)). Viral RNA-dependent RNA polymerases (RdRp) are essential for replication of RNA viruses and represent important drug targets. Despite recent progress in the field (Liang, B. et al. Cell 162, 314-327 (2015); Noton, S. L., et al. PLoS pathogens 8, e1002980 (2012); Pflug, A., et al. Nature 516, 355-360 (2014); Reich, S. et al. Nature 516, 361-366 (2014); Deval, J. et al. PLoS pathogens 11 (2015)), the expression of active recombinant RdRp enzymes of negative-sense RNA viruses remains challenging. Influenza (Flu) and vesicular stomatitis (VSV) viral RdRp complexes have been successfully crystallized and RNA synthesis of these protein complexes, including the RSV complex, can be monitored in biochemical assays (Noton, S. L., et al. supra; Deval, J. et al. supra; Reich, S., et al. Nucleic acids research 45, 3353-3368 (2017); Jin, Z., et al. PloS one 8, e68347 (2013); Morin, B., et al. The EMBO journal 31, 1320-1329 (2012)).

However, methods for the study of EBOV RdRp have yet to be devised. Efforts to identify and validate inhibitors of this enzyme still rely on biochemical surrogate systems (Warren, T. K. et al. Nature 531, 381-385 (2016)). A nucleotide analogue (GS-5734) has been shown to be a potent inhibitor of EBOV infection in cell culture and provided post-exposure protection in non-human primates. Biochemical evidence for a targeted action against EBOV RdRp was generated with the RSV RdRp presumably due to lack of availability of an active EBOV RdRp.

Here we report the expression, purification, and biochemical characterization of an active, recombinant EBOV RdRp complex. Multiprotein complexes derived from negative-sense RNA viruses RSV5 and influenza B (FluB) (Pflug, A., 2014, supra; Reich, S. et al. 2014, supra), and monomeric polymerases derived from positive-sense RNA viruses hepatitis C virus (HCV) and Zika virus served as benchmarks (D'Abramo, C. M., et al. The Journal of biological chemistry 281, 24991-24998 (2006); Hou, S. et al. J Virol (2017)).

We designed an expression vector that is based on constructs successfully used to produce the trimeric Influenza RdRp complex in insect cells (Pflug, A., 2014, supra; Reich, S. et al. 2014, supra). The three components of this complex (PA, PB1, and PB2) are expressed from the same promoter to yield a single polyprotein, which is cleaved by the co-expressed tobacco etch virus (TEV) protease at engineered cleavage sites. The identity of purified proteins was confirmed by mass spectrometry analysis (MS). Purification of the trimeric complex is accomplished through affinity chromatography (FIG. 1a, Table 1).

TABLE 1
Detailed information regarding viral RdRp production and RNA synthesis assays.
VIRUS

| family | Flaviviridae | | Orthomyxoviridae | Pneumoviridae | Filoviridae |
|---|---|---|---|---|---|
| genus | Hepacivirus | Flavivirus | Influenza B | Orthopneumovirus | Ebolavirus |
| species | Hepatitis C | Zika virus | Influenza B virus | Human respiratory | Zaire ebolavirus |
| | virus | | | syncytial virus | |
| virus | Hepatitis C | Zika | Influenza B | human respiratory | Ebola |
| | | | | syncytial virusA2 | |
| strain or | AJ238799 | ZikaSPH2015: | B/Memphis/13/03 | A2 | KR534507.1 |
| isolate | | KU321639 | | | |
| protein | CAB46677.1 | ALU33341.1 | PA: AAU94844 | P: AAB59853 | VP35: AKG65095 |
| | | | PB1: AAU94857 | L: AAA84898 | VP30: AKG65100 |
| | | | PB2: AAU94870 | | L: AKG65102 |

EXPRESSION CONSTRUCT, PROTEIN PRODUCTION

| coding region | CAB46677.1 | Synthetic DNA codon-optimized for expression in insect cells (GenScript) | | | | |
|---|---|---|---|---|---|---|
| | 2420-3010 | | | | | |
| design | Polyprotein: | Polyprotein: | | | | |
| details | 2420-2989 | 2525-3416 | | | | |
| | [SMS-RPR] | [GETL-LGEE] | | | | |
| vector | pET21b | pFastBac1, Bacmid | | | | |
| composition | NS5b | NS5 (domains): | PA | P | vp35 | vp35 |
| | Δ21 | Methyltransferase | PB1 | L | vp30 | L |
| | C-terminus | linker | PB2 | | L | |
| | | RdRp | | | | |

| | | |
|---|---|---|
| host | E. coli | Spodoptera frugiperda (Sf9) |

PURIFICATION

| AFFINITY TAG | Histidine | Streptavidin | Streptavidin | Histidine | | |
|---|---|---|---|---|---|---|
| terminus | C | N | C | N | | |
| location | NS5b | NS5 | PB2 | P | vp35 | vp35 |

| | | | | |
|---|---|---|---|---|
| WASH | NaCl, 500 mM | NaCl, 1000 mM | NaCl, 1000 mM | NaCl, 500 mM |
| BUFFER | 40 cv | 60 cv | 60 cv | 60 cv |
| Tween 20 | | | | 0.01, % |
| Concentrator | 30 kDa | 50 kDa | 30 kDa | 100 kDa |

| | |
|---|---|
| STORAGE BUFFER | Tris, 50 mM, pH 8, NaCl, 75 mM, TCEP, 2 Mm, Glycerol, 50%, final |

RNA SYNTHESIS REACTION MIXTURE

| buffer | Tris, 25 Mm, Ph 8, NaCl, 20 mM, TCEP, 2 mM | | | | | |
|---|---|---|---|---|---|---|
| RNA substrate | Template, 1 μM, Primer, 200 μM | | | | | |
| NTP substrate | 1 μM | 1 μM | 0.1 or 1 μM | 10 μM | 100 μM | |
| enzyme | 0.4 μM | 0.1 μM | 0.1 μM | 0.2 μM | 2 uL | 2uL |

| | | | | |
|---|---|---|---|---|
| reaction | MgCl₂, | MnCl₂, | MnCl₂, | MgCl₂, |
| start | 5 mM | 2.5 mM | 2.5 mM | 5 mM |

| | |
|---|---|
| stop | Formamide, 95%, EDTA, 25 mM |

We used the same approach to produce the dimeric P:L complex of RSV (FIG. 1b), while others express the two proteins from different promoters (Noton. S. L. et al. 2012, supra, Deval, J. et al. 2015). The requirements for the expression of an active Ebola RdRp complex are unknown. While the L protein is essential for RNA synthesis, the nucleoprotein NP and viral proteins VP30 and VP35 have been considered as possible additional factors (Muhlberger, E., et al. J Virol 73, 2333-2342 (1999)). NP is primarily involved in RNA binding, likely independent of the L protein. While the possible contribution of VP30 remains to be defined, VP35 is considered as a functional equivalent of the RSV-associated P protein ((Muhlberger, E., et al., supra). Hence, we designed vectors that express the two components L and VP35, or the three components L, VP30, and VP35 from a single promoter. Protein expression in insect cells employed here proved to be the system of choice for the production of both monomeric RdRp from positive-sense RNA viruses and multimeric RdRp from negative-sense RNA viruses. The purity of the FluB RdRp complex is comparable to the previously described method (FIG. 1a) (Pflug, A., 2014, supra; Reich, S. et al. 2014, supra). The adaption of this method to produce RSV L:P RdRp complex resulted in a considerably pure protein preparation with only small amounts of heat shock proteins HSP70 and HSP90 from insect cells (FIG. 1b). To remove smaller proteins that are not bound to the complex, the RSV and EBOV protein preparations were concentrated with 100 kDa molecular weight cut-off membranes prior to PAGE analysis. However, Ebola RdRp complexes co-purified with higher amounts of Hsp70 and Hsp90 despite the presence of 0.5 M NaCl (FIG. 1c, Table 1). Co-purification with heat shock proteins has been also reported previously by others for the protein preparations of RSV L:P complexes (Noton, S. L. et al. 2012, supra). In fact, Hsp90 is implicated in L protein stability and RdRp complex formation (Connor, J. H., et. al. Virology 362, 109-119 (2007); Katoh, H. et al. Journal of Virology 91 (2017); Smith, D. R. et al. Antiviral research 87, 187-194 (2010)). Nevertheless, the presence of insect heat shock proteins precluded an accurate estimation of the protein complex concentration in the Ebola RdRp samples. Despite the predicted structural similarity between RSV and EBOV RdRp (Liang, B. et al., supra; Warren, T. K. et al., supra), the dimeric L:VP35 and trimeric L; VP35:VP30 EBOV RdRp complexes express at considerably lower levels then RSV P:L complex. Expression of monomeric RdRp of Zika and HCV is shown in FIGS. 1d and 1e, respectively.

FIG. 1. SDS PAGE migration patterns of purified viral proteins. Arrows point to the bands containing the relevant full-length proteins identified by mass spectrometry. “m” represents the marker (a) ˜5 μg of FluB protein (lane 1) and a 20-fold dilution of the same sample (lane 20). The diluted sample shows the three components PA, PB1, and PB2 of the trimeric protein complex. (b) The RSV preparation shows the L and P proteins of the dimeric complex (lane P+L). (c) The Ebola preparation shows viral proteins L and VP35 (lane L:VP35), and L, VP35, and VP30 (lane L:VP35:VP30). Cellular proteins Hsp70 and Hsp90 have also been identified. (d) and (e) ˜3-4 μg of HCV and Zika proteins, respectively, were loaded on the gel and analyzed as in (a).

### Example 2: Assessment and Optimization of RNA Synthesis Activity

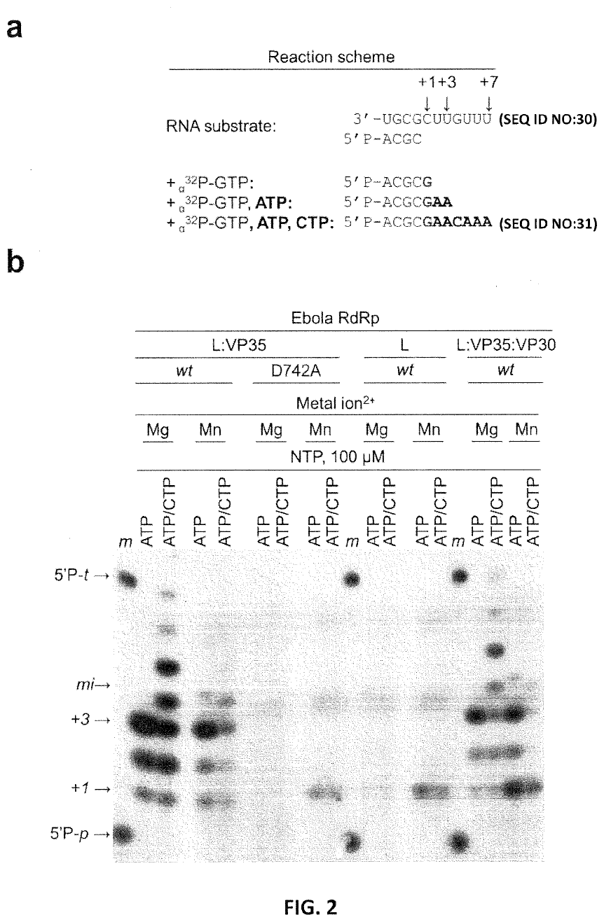

Based on the related nature of the RSV and EBOV RdRp complexes, we monitored RNA synthesis activity using a tested RSV-derived, RNA model primer/template (P/T) substrate (Deval, J. et al., 2015, supra) (FIG. 2). The primer is phosphorylated at its 5′-end and contains four nucleotides that are complementary to the 3′-end of a 7-mer template (FIG. 2a). The template permits incorporation of a radio-labelled nucleotide at position +1, and, depending on the available NTPs, formation of an intermediate product at position +3, and a full-length product at position +7. Activity was tested with the potential catalytic, divalent metal ions Mg²⁺ and Mn²⁺, respectively. In the presence of Mg²⁺, the dimeric complex L:VP35 shows the expected products at position +1, +3, and +7 (FIG. 2b). We obtained essentially the same data with the trimeric complex L:VP30:VP35, indicating that VP30 is not essentially required for RNA synthesis. Mn²⁺ is less efficient in catalyzing the reaction, and, under these conditions, RNA synthesis is more prone to misincorporations. A faint band corresponding to G:A misincorporation at position +4 was detected only in the presence of Mn²⁺ (FIG. 2b, panel L:VP35, sub-panel wt, lanes ATP).

Several conserved motifs characterize the environment around the active site of RdRp enzymes (Jacome, R., et al. PloS one 10, e0139001 (2015); Poch, O., et al. The EMBO journal 8, 3867-3874 (1989)). Motif C (Table 2, sequence alignment) is responsible for coordinating metal ions during catalysis (Ng, K. K., et al. Current topics in microbiology and immunology 320, 137-156 (2008)).

TABLE 2

| | |
|---|---|
| sequence | Structure-based alignment of viral RdRp (Chimera, UCSF: Pettersen, E.F. et al. |
| alignment | Journal of computational chemistry 25, 1605-1612 (2004)), top panel. Sequence- |
| | based alignment of vesicular stomatitis, RSV and Ebola L proteins (T-coffee- |
| | Notredame, C., et al. Journal of molecular biology 302, 205-217 (2000)). Sequences |
| | for Motif F3 of the RdRp the listed virus: HCV, Zika, Polio, FluB, VSV are |
| | designated as SEQ ID NO: 1, 2, 3, 4, and 5, respectively. Sequences for Motif A of |
| | the listed virus: HCV, Zika, Polio, FLuB, VSV are designated as SEQ ID NO: 6, 7, 8, |
| | 9, and 10, respectively. Sequences for Motif B of the listed virus: HCV, Zika, Polio, |
| | FLuB, VSV are designated as SEQ ID NOs: 11, 12, 13, 14, and 15, respectively. |
| | Sequences for Motif C of the listed virus: HCV, Zika, Polio, FLuB, VSV are |
| | designated as SEQ ID NOs: 16, 17, 18, 19, and 20, respectively. |
| | Bottom panel. The alignments were rendered using ESPript-Robert, X. & Gouet, P. |
| | Nucleic acids research 42, W320-324 (2014). Numbers above the alignment refer to |
| | the HCV residues that are in contact with the 2′-OH of the incoming nucleotide |
| | (pdb:4WTA-Appleby, T.C. et al. Science 347, 771-775 (2015)). Number below the |
| | alignment (742) refers to the Ebola L protein residue that was mutated to alanine. |
| | Sequences for Motif F3 of the RdRp the listed virus: VSV, RSV, Ebola are |
| | designated as SEQ ID NOs: 5, 21, and 22, respectively. Sequences for Motif A of the |
| | listed virus: VSV, RSV, Ebola are designated as SEQ ID NOs: 10, 23, and 24, |
| | respectively. Sequences for Motif B of the listed virus: VSV, RSV, Ebola are |
| | designated as SEQ ID NOs: 15, 25, and 26, respectively. Sequences for Motif C of |
| | the listed virus: VSV, RSV, Ebola are designated as SEQ ID NOs: 20, 27, and 28, |
| | respectively. |
| | 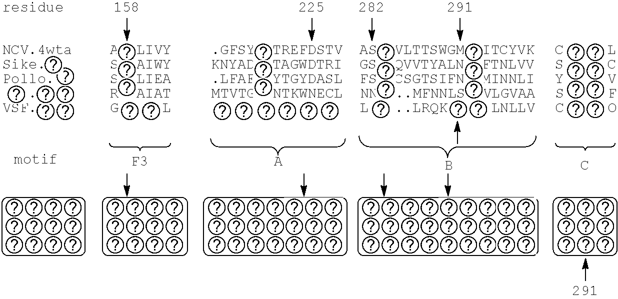 |

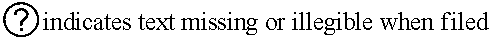

Amino acid substitutions within the Motif C generally result in an inactive enzyme (Noton et al. supra, Fix, J., et al. The open virology journal 5, 103-108 (2011); Lu, G. et al. Antimicrob Agents Chemother 61 (2017)). In particular, changing the first aspartate of the GDD/N sequence within Motif C was shown to cause ablation of the nucleic acid synthesis in some RdRp (Lohmann, V., et al. J Virol 71, 8416-8428 (1997); Vazquez, A. L., et al. Journal of virology 74, 3888-3891 (2000)). The corresponding residue in EBOV RdRp is D742 (Table 2). We demonstrate that the EBOV D742A mutant enzyme lacks the ability to extend the primer in the presence of Mg²⁺ (FIG. 2b). In the presence of Mn²⁺, a faint band is seen at position +1 that is indicative of poor incorporation of the radio-labelled nucleotide. The same result was obtained with the L protein that was expressed in the absence of VP30 and VP35.

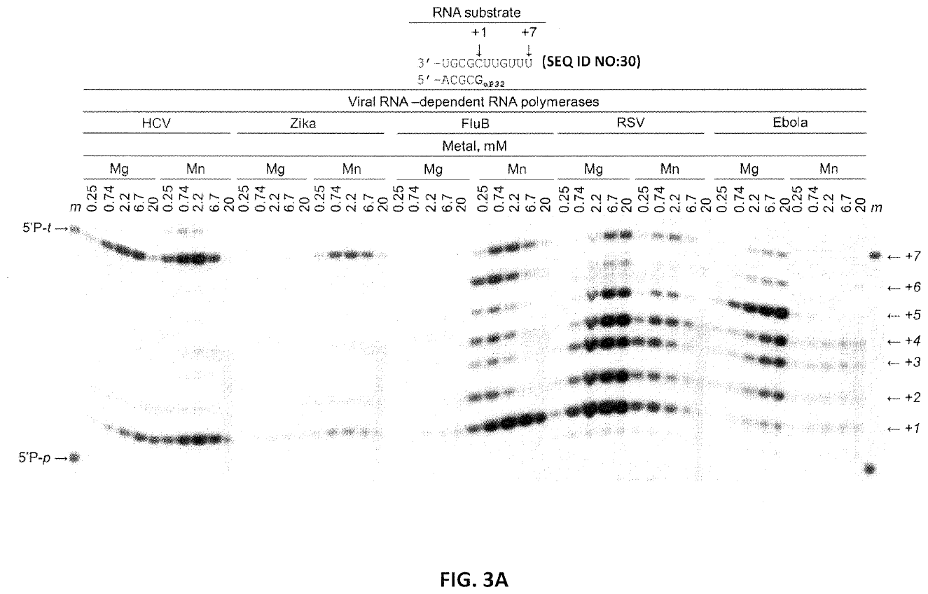

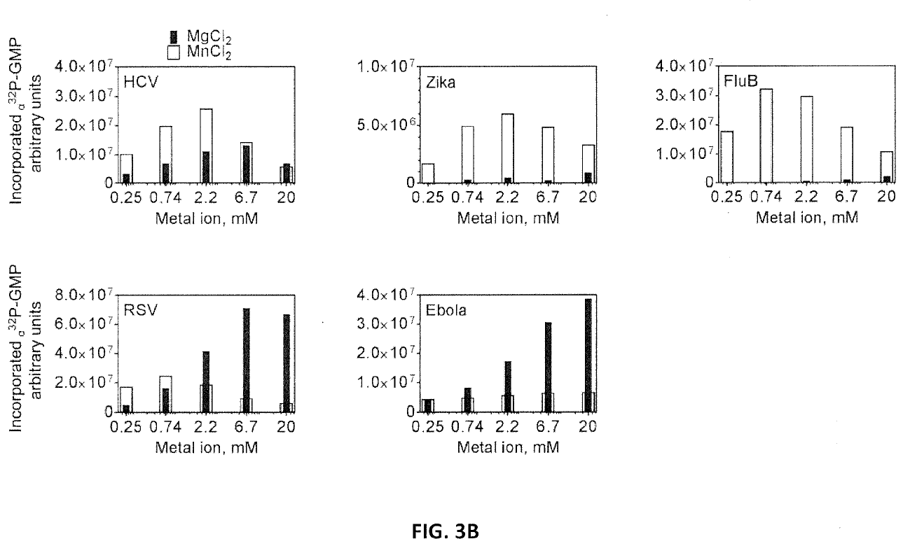

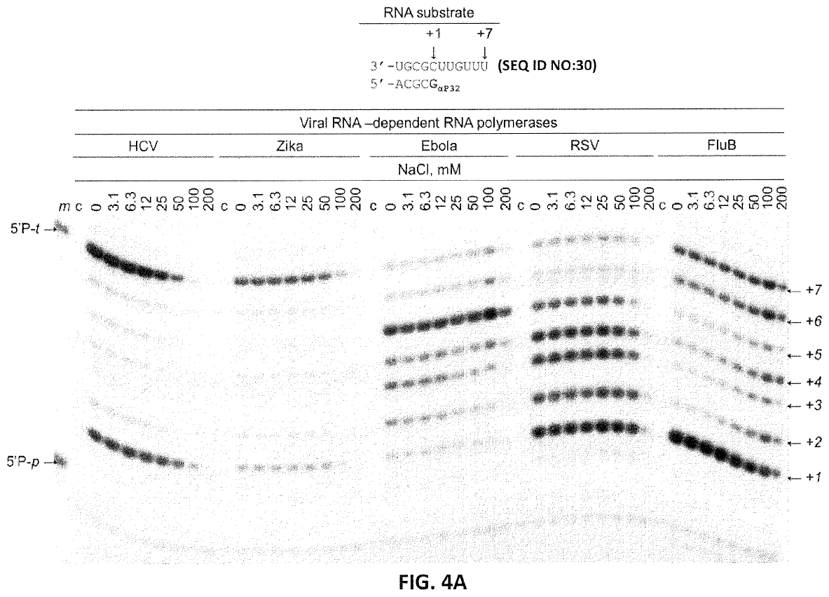

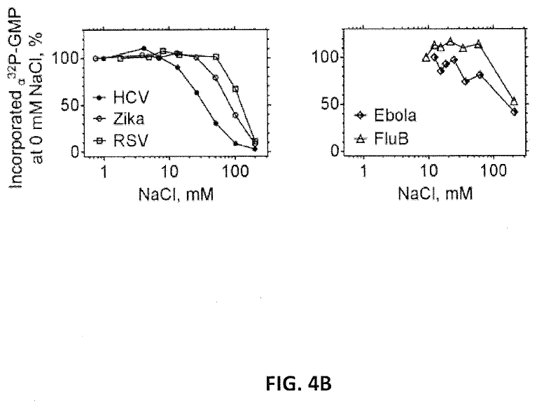

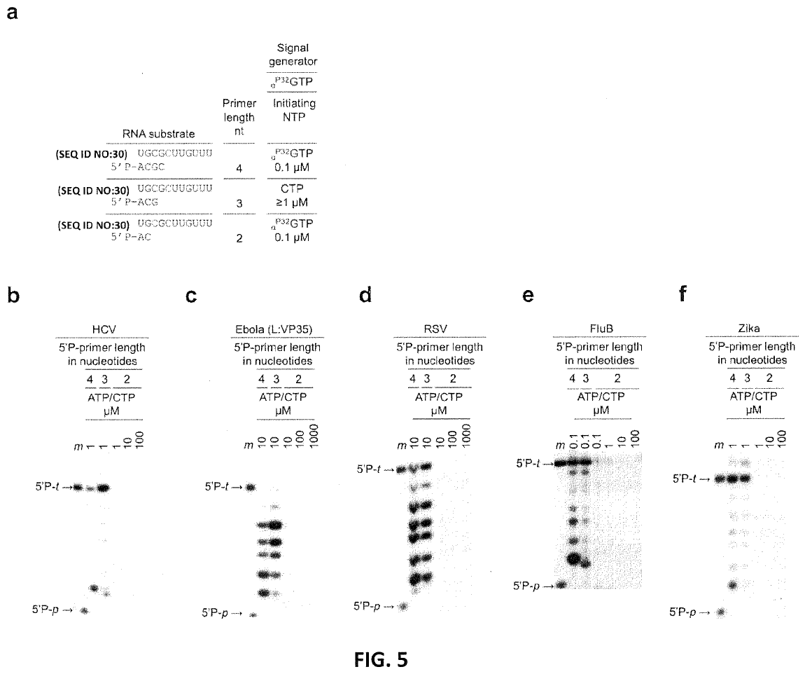

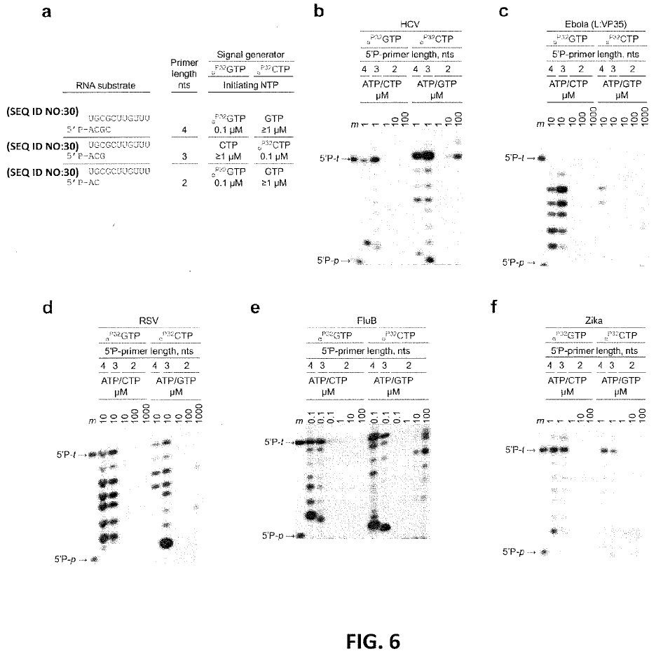

For comparative analyses, we optimized assay conditions for RNA synthesis for each of the enzymes used in this study. Zika and FluB RdRps showed optimal activity in the presence of 2.5 mM MnCl₂, while HCV, RSV and Ebola RdRp showed optimal activity at 5 mM MgCl₂ (FIGS. 3 and 4). 20 mM NaCl was used for all enzyme reactions, which is based on the limitations seen with HCV RdRp. We also considered the length of the primer as an additional reaction parameter and determined the minimum length required for RNA synthesis (FIG. 5). We utilized 5′-phosphorylated primers composed of four, three, and two nucleotides (FIG. 5a). Our data show that a tri-nucleotide primer was sufficient for efficient RNA synthesis by each of the aforementioned five viral polymerases, while a di-nucleotide primer did not yield significant amounts of product (FIG. 5b-f). This observation is consistent with the proposed transition from a fragile, distributive initiation process to a more stable and processive elongation mode in HCV (Harms, D. et al. The Journal of biological chemistry 285, 32906-32918 (2010); Appleby, T. C. et al. Science 347, 771-775 (2015); Dutartre, H., et al. The Journal of biological chemistry 280, 6359-6368 (2005)). Our data point to a similar transition in Zika, FluB, RSV, and Ebola RdRp (FIG. 3c-f). It is also evident that HCV, Zika, and FluB RdRps yields predominantly full-length+7 RNA products, while Ebola and to a certain degree also RSV RdRp shows a ladder of products up to +5 and minor products at +6 and +7, which points to relative deficits in processive RNA elongation. Depending on the primer length used in our reaction, the initiating nucleotide may also be the radioactively-labelled nucleotide (FIG. 5a). RNA synthesis is here limited by the sub-micromolar concentration of _{α}^{32P}-GTP. Under these conditions HCV RdRp does not initiate RNA synthesis from a di-nucleotide primer (FIG. 5b). Moreover, increasing concentration of nucleotides for subsequent incorporations (positions +2, +3 (ATP) and +4 (CTP)) fails to rescue RNA synthesis. However, RNA synthesis is efficiently initiated with non-labeled nucleotides that can be used at higher concentrations (FIG. 6). Under these conditions HCV RdRp initiates RNA synthesis from a dinucleotide primer at 100 μM concentrations of the initiating nucleotide (GTP) (FIG. 6b, right panel). FluB shows a very similar pattern (FIG. 6e, right panel). None of the remaining enzymes was capable of initiating dinucleotide-primed RNA synthesis to a comparable extent under similar conditions.

FIG. 2. Limited RNA synthesis by Ebola RdRp. 15% denaturing PAGE shows the 5′P-RNA primer and its extension products. (a) The reaction scheme shows the nucleotide mixtures that yield one, three or seven nucleotide extensions. (b) RNA synthesis in the presence of Mn²⁺ or Mg²⁺ with the binary complex L:VP35 (wt, wild type and the active site mutant D742A), the monomeric L protein and the ternary complex (L:VP35:VP30). Lane m, 5′-³²P-labeled primer (5′P-p→) and template (5′P-t→) markers. mi→, product of misincorporation at position +4. SEQ ID NO:30 provides the sequence of the template used in the assay for measuring RdRp activity of recombinant RdRp of Ebola virus. SEQ ID NO: 31 provides the sequence of the nucleic acid synthesized by extension of the primer by RdRp activity.

FIG. 3. RNA synthesis by viral RdRp as a function of di-valent metal ions concentration. FIG. 3A 15% denaturing PAGE migration pattern of 5′P-RNA primers extended through incorporation of nucleotides. RNA substrate consists of a 5′-phosphorylated primer and 11-nucleotide template. The template permits incorporation of a radio-labelled nucleotide (_{α}³²P-GTP, G_{αP32}), which effectively labels the primer-extended products of the RNA synthesis. The reaction mixture contains ATP and CTP (Table 1) nucleotide substrates which allows synthesis of a fully extended primer product position +7. Lane m, PAGE migration pattern of 5′-³²P-labeled primer (5′P-p→) and template (5′P-t→) markers. FIG. 3B Graphical representation of the extent of RNA synthesis by viral RdRp as a function of di-valent metal ions concentration.

FIG. 4. RNA synthesis by viral RdRp as a function of mono-valent metal ion concentration. (a) 15% denaturing PAGE migration pattern of 5′P-RNA primers extended through incorporation of nucleotides. RNA substrate consists of a 5′-phosphorylated primer and 11-nucleotide template. The template permits incorporation of a radio-labelled nucleotide (_{α}³²P-GTP, G_{αP32}), which effectively labels the primer-extended products of the RNA synthesis. The reaction mixture contains ATP and CTP (Table 1) nucleotide substrates which allow synthesis of a fully extended primer product position +7.

Lane m, PAGE migration pattern of 5′-³²P-labeled primer (5′P-p→) and template (5′P-t→) markers. Lane c, RNA synthesis by viral RdRp in the absence of di-valent metal ions. (b) Graphical representation of the extent of RNA synthesis by viral RdRp as a function of mono-valent metal ion concentration.

FIG. 5. RNA synthesis as a function of primer length. (a) RNA substrates are shown for different primer lengths: 4, 3, and 2. The reaction conditions are such that the concentration of the first nucleotide to be incorporated at the 3′-end of the primer (initiating NTP) changes as a function of primer length (nts, nucleotides) used in the reaction mixture. Template positions at which radio-labeled nucleotide is incorporated are illustrated in red. (b-f) RNA synthesis by viral RdRp as a function of 5′-phosphorylated primer length. 15% denaturing PAGE migration pattern of 5′-phosphorylated primers of various lengths extended through incorporation of nucleotides. Lane m, 5′-³²P-labeled primer (5′P-p→) and template (5′P-t→) markers.

FIG. 6. RNA synthesis as a function of primer length. (a) RNA substrates are shown for different primer lengths: 4, 3, and 2. The reaction conditions are such that the concentration of the first nucleotide to be incorporated at the 3′-end of the primer (initiating NTP) changes as a function of primer length (nts, nucleotides) used in the reaction mixture. (b-f) RNA synthesis by viral RdRp as a function of 5′-phosphorylated primer length. 15% denaturing PAGE migration pattern of 5′-phosphorylated primers of various lengths extended through incorporation of nucleotides including radio-labelled _{α}^{32P}-GTP (left panel) or _{α}^{32P}-CTP (right panel). Lane m, 5′-³²P-labeled primer (5′P-p→) and template (5′P-t→) markers.

### Example 3: Selectivity of Nucleotide Incorporation

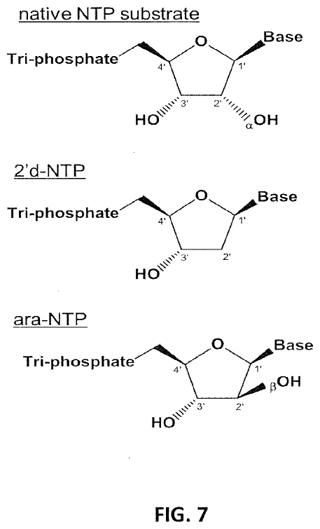

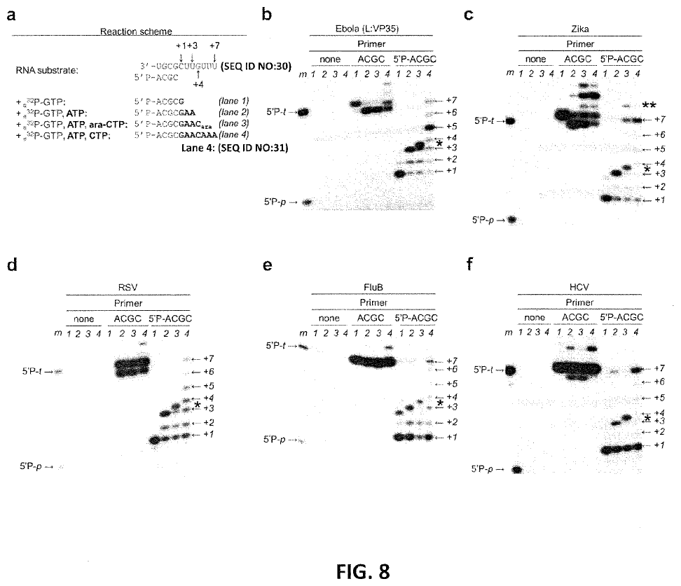

RNA polymerases are expected to interact with the 2′-OH group of the incoming NTP (Appleby, T. C. et al. Science 347, 771-775 (2015)). Hence, we tested the ability of the Ebola enzyme to accommodate nucleotide analogues 2′β-hydroxy-cytidine-5′-triphosphate (ara-CTP) and 2′-deoxy-cytidine-5′-triphosphate (2′-dCTP) that are modified at this position. The 2′-OH group is in the “up” β-conformation in ara-CTP as opposed to the “down” a-conformation in natural CTP pools, while the 2′-OH group is absent in 2′-dCTP (FIG. 7). Incorporation of ara-CTP or 2′-dCTP is enabled at template position +4 (FIG. 8a). Depending on the nucleotide mixture, products are expected at positions +1, +3, +4, and +7. Reactions were performed without a primer, with a non-phosphorylated primer, and with a 5′-phosphorylated primer as described above (FIG. 8b-f). No radio-labeled products were detected in the absence of a primer. Although we identified labelled reaction products with a non-phosphorylated primer, the nature of this activity remains elusive. Product formation is here rather independent of the template sequence. Similar patterns are seen with each of the five enzymes, suggesting that the lack of a 5′-phosphate group and not the nature of the enzyme causes these effects. In contrast, data obtained with the 5′-phosphorylated primer yielded the expected reaction products (FIG. 8b-f). The product at position +1 is a 5-mer with incorporated _{α}³²P-GTP (lane 1), and the product at position +3 is indicative of a 7-mer that shows incorporation of ATP at positions +2 and +3 (lane 2). The reaction with ara-CTP shows a product that migrates between positions +3 and +4 (lane 3), and the lack of longer reaction products demonstrates effective chain-termination. Longer products, including the full-length product are seen in the control in which ara-CTP was replaced with CTP (lane 4). With regards to the incorporation of ara-CTP, RSV, FluB, and HCV enzymes show the same pattern as the Ebola enzyme (FIG. 8b, d-f). In contrast, Zika RdRp incorporates ara-CTP and yields in addition the full-length product at position +7 (full-length) and a product at position +8 that is likely a result of terminal transferase activity (FIG. 8c). These data are indicative of incomplete chain-termination. Although essential to yield interpretable data, the role of the 5′-phosphate moiety of the RNA primer merits further investigation.

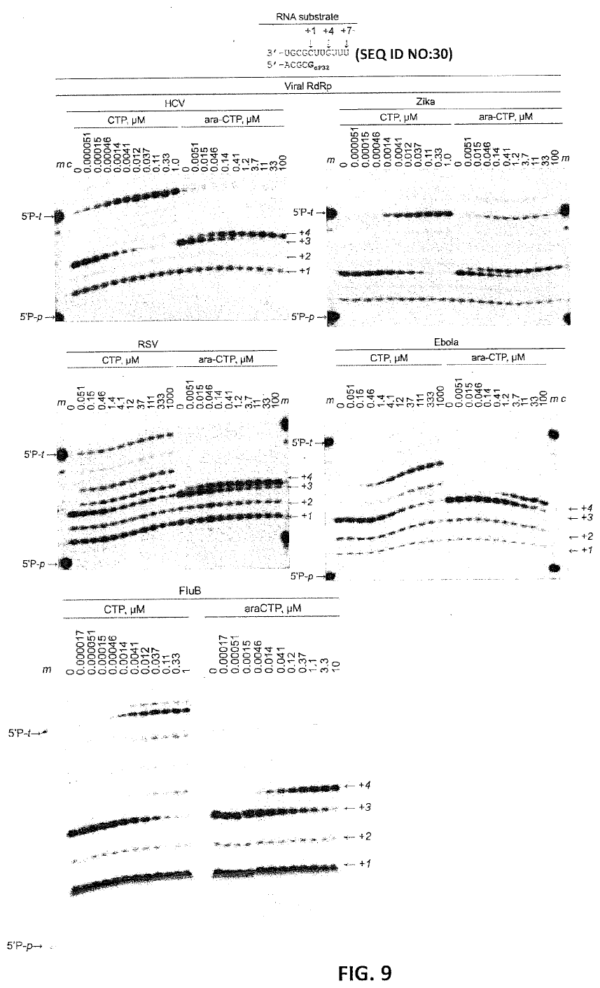

The above assay was modified to measure selective incorporation of the natural CTP substrate over ara-CTP and 2′-dCTP, respectively (FIG. 9-11). The selectivity values follow the order HCV >FluB >Zika >EBOV >RSV and HCV >Zika >EBOV >RSV >FluB, respectively (Table 3 and Table 4). FluB RdRp shows the lowest selectivity value with respect to 2′-dCTP, while it ranks second with respect to ara-CTP. A -100-fold spread in 2′d-CTP and ara-CTP selectivity values exhibited by the five viral RdRp enzymes illustrates virus-specific nucleotide substrate requirements during RNA genome replication.

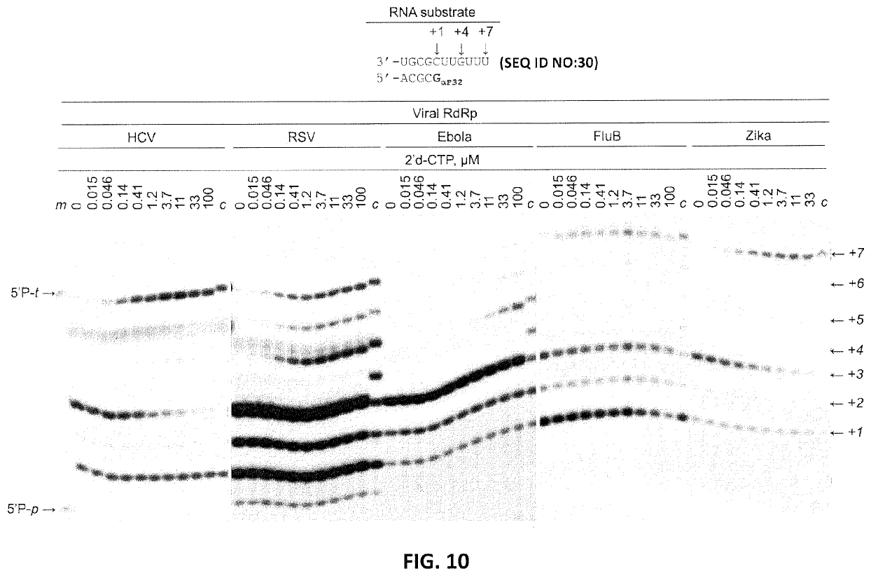

TABLE 3
Nucleotide substrate analogue selectivity of viral RNA-dependent
RNA polymerases. (see FIGS. 7 and 9 for an example of raw data,
FIG. 10 for overall data quality and analysis and Table 4 for
complete data summary and calculation of the selectivity values;
values have been calculated on the basis of a 12-data point
experiment repeated at least 3 times for natural substrate and
the two substrate analogues for each of the five enzymes).

Nucleotide substrate analogue

| | | |
|---|---|---|
| Viral | ara-CTP | 2′-dCTP |

| | |
|---|---|
| RdRp | Selectivity (fold) |

| | | |
|---|---|---|
| HCV | 100 | 654 |
| Zika | 8 | 90 |
| Ebola | 3 | 73 |
| RSV | 0.7 | 24 |
| FluB | 23 | 6 |

TABLE 4
Determination of the selectivity values for ara-CTP and 2′d-CTP nucleotide substrate analogues. (complete
summary; see FIGS. 7 and 9 for an example of raw data, FIG. 10 for overall data quality and analysis; values
have been calculated on the basis of a 12-data point experiment repeated at least 3 times (n=) for natural
substrate and the two substrate analogues for each of the five enzymes). Eff., efficiency of nucleotide substrate
incorporation during RNA synthesis; calculated of a viral RdRp for a given nucleotide substrate analogue; calculated
as the ratio of the efficiency of CTP incorporation to the efficiency of the respective nucleotide substrate
analogue incorporation. Std. err., standard error associated with the fit. % err., percent error.

| CTP | | | 2′dCTP | | | | ara-CTP | | | |
|---|---|---|---|---|---|---|---|---|---|---|
| Vₘₐₓ | Kₘ | | Vₘₐₓ | Kₘ | | | Vₘₐₓ | Kₘ | | |
| p. frac. | μM | Eff. | p. frac. | μM | Vₘₐₓ/Kₘ | Sel. | p. frac. | μM | Vₘₐₓ/Kₘ | Sel. |

HCV

| | n = 4 | | | n = 3 | | | | n = 3 | | | |
|---|---|---|---|---|---|---|---|---|---|---|---|
| | 0.97 | 0.00060 | 1617 | 0.84 | 0.34 | 2.5 | 654 | 0.95 | 0.059 | 16 | 100 |
| Std. err. | 0.0083 | 0.000034 | | 0.014 | 0.032 | | | 0.01 | 0.0040 | | |
| % err. | 1 | 6 | | 2 | 9 | | | 1 | 7 | | |

Zika

| | n = 3 | | | n = 3 | | | | n = 3 | | | |
|---|---|---|---|---|---|---|---|---|---|---|---|
| | 0.96 | 0.0032 | 300 | 0.83 | 0.25 | 3 | 90 | 0.95 | 0.026 | 37 | 8 |
| Std. err. | 0.0064 | 0.00012 | | 0.012 | 0.021 | | | 0.011 | 0.0019 | | |
| % err. | 1 | 4 | | 1 | 8 | | | 1 | 7 | | |

Ebola

| | n = 3 | | | n = 3 | | | | n = 3 | | | |
|---|---|---|---|---|---|---|---|---|---|---|---|
| | 0.82 | 1.2 | 0.68 | 0.59 | 63 | 0.01 | 73 | 0.77 | 3.2 | 0.24 | 3 |
| Std. err. | 0.014 | 0.11 | | 0.022 | 7.7 | | | 0.015 | 0.27 | | |
| % err. | 2 | 9 | | 4 | 12 | | | 2 | 8 | | |

RSV

| | n = 3 | | | n = 3 | | | | n = 3 | | | |
|---|---|---|---|---|---|---|---|---|---|---|---|
| | 0.70 | 0.049 | 14 | 0.27 | 0.45 | 0.60 | 24 | 0.74 | 0.035 | 21 | 0.68 |
| Std. err. | 0.0088 | 0.0042 | | 0.0053 | 0.048 | | | 0.012 | .0038 | | |
| % err. | 1 | 9 | | 2 | 11 | | | 2 | 11 | | |

FluB

| | n = 6 | | | n = 7 | | | | n = 5 | | | |
|---|---|---|---|---|---|---|---|---|---|---|---|
| | 0.88 | 0.0038 | 232 | 0.38 | 0.010 | 38 | 6 | 0.77 | 0.077 | 10 | 23 |
| Std. err. | 0.012 | 0.003 | | 0.0078 | 0.0015 | | | 0.012 | 0.0061 | | |
| % err. | 1 | 8 | | 2 | 15 | | | 2 | 8 | | |

FIG. 7. Chemical structures of cytidine nucleotide substrate analogues used in the study.

FIG. 8. RNA synthesis and inhibition. (a) Reaction scheme for the assay: the primer is extended by one, three, four or seven nucleotides depending on the nucleotide mixture as indicated. (b-f) 15% denaturing PAGE migration pattern of products of RNA synthesis in the absence (left panel), in the presence of non-phosphorylated primers (middle panel) and 5′-phosphorylated primers (right panel). Lane m, PAGE migration pattern of 5′-³²P-labeled primer (5′P-p→) and template (5′P-t→) markers. Asterisk (*) indicates the migration pattern of ara-CMP-terminated primers (lane 3). SEQ ID NO:30 (5′-UUUGUUCGCGU-3′) provides the sequence of the template used in the assay for measuring RdRp activity of recombinant RdRp of HCV, RSV, Ebola, FluB, and Zika virus. SEQ ID NO: 31 (5′-ACGCGAACAAA-3′) provides the sequence of the nucleic acid synthesized by extension of the primer by RdRp activity.

FIG. 9. RNA synthesis by viral RdRp as a function of incorporation of ara-CMP at position +4 with respect to RNA template. 20% denaturing PAGE migration pattern of products of reactions containing titrated nucleotide substrate concentrations of either CTP or ara-CTP. Lane m, PAGE migration pattern of 5′-³²P-labeled primer (5′P-p→) and template (5′P-t→) markers. SEQ ID NO:30 provides the sequence of the template used in the assay for measuring RdRp activity of recombinant RdRp of HCV, RSV, Ebola, FluB, and Zika virus.

FIG. 10. RNA synthesis by viral RdRp as a function of incorporation of 2′d-CMP at position +4 with respect to RNA template. 20% denaturing PAGE migration pattern of products of reactions containing titrated nucleotide substrate concentrations of either CTP or ara-CTP. Lane m, PAGE migration pattern of 5′-³²P-labeled primer (5′P-p→) and template (5′P-t→) markers. SEQ ID NO:30 provides the sequence of the template used in the assay for measuring RdRp activity of recombinant RdRp of HCV, RSV, Ebola, FluB, and Zika virus.

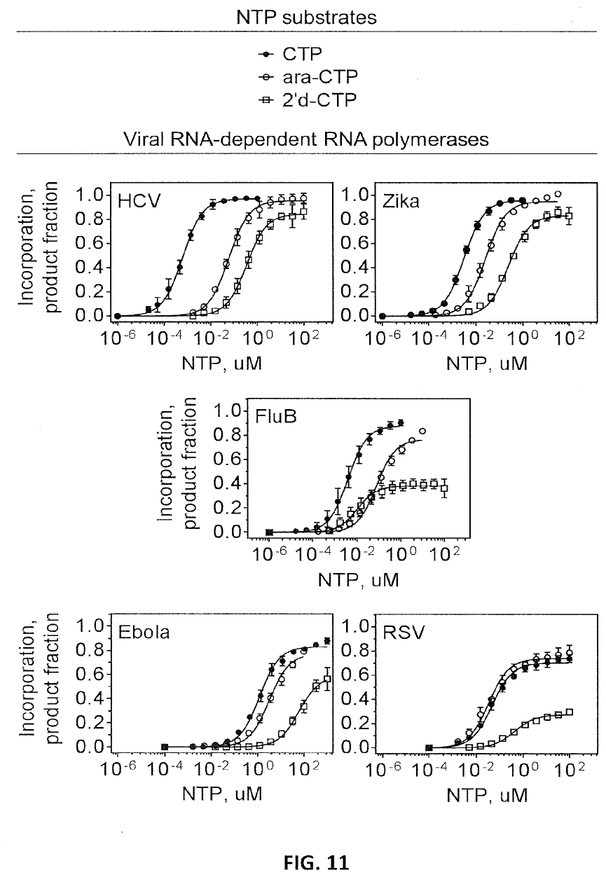

FIG. 11. Data quality and analysis of incorporation of ara-CMP and 2′d-CMP during RNA synthesis by viral RdRp. Error bars represent standard deviation of the data points determined on the basis of at least three independent experiments. Data points were fit to a Michaelis-Menten equation using Prism software (GrapPad). Michaelis-Menten parameters calculated from the fits are reported in Table 3 and Table 4.

### Example 4: Expression and Characterization of Negative Sense ss-RNA Viruses

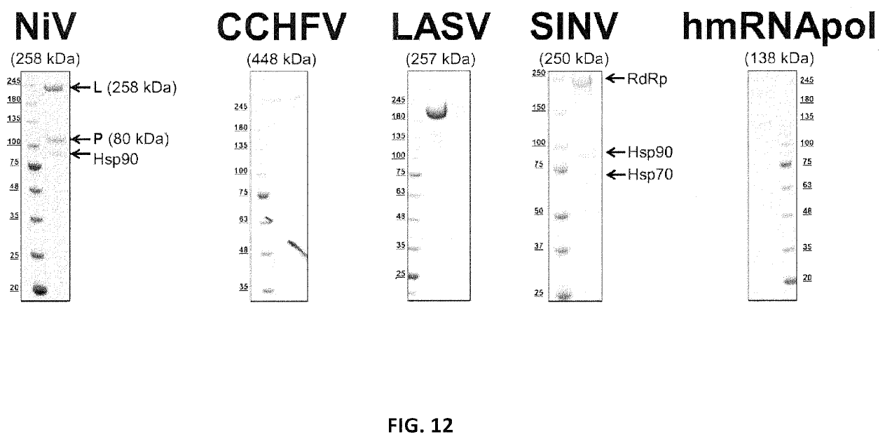

FIG. 12. Expression of new negative-sense RNA viral RNA-dependent RNA polymerases (RdRp) and human mitochondrial RNA polymerase employing the Baculovirus expression system as described herein. The expression of Crimean-Congo haemorrhagic fever (CCHFV), LASV, SINV RdRp has not been previously reported. The expression of hmRNApol using a eukaryotic expression system has not been reported. The expression of Nipah Virus (NiV) RdRp using a single promoter has not been reported. The single promoter expression system employed here results in a greater amount of the P protein as compared with dual promoter system reported by Jordan et al., 2018, PLoS Pathog 14(2): e1006889. Of note, the CCHFV pol which is extremely large for a single polypeptide chain was expressed successfully using the expression system described herein.

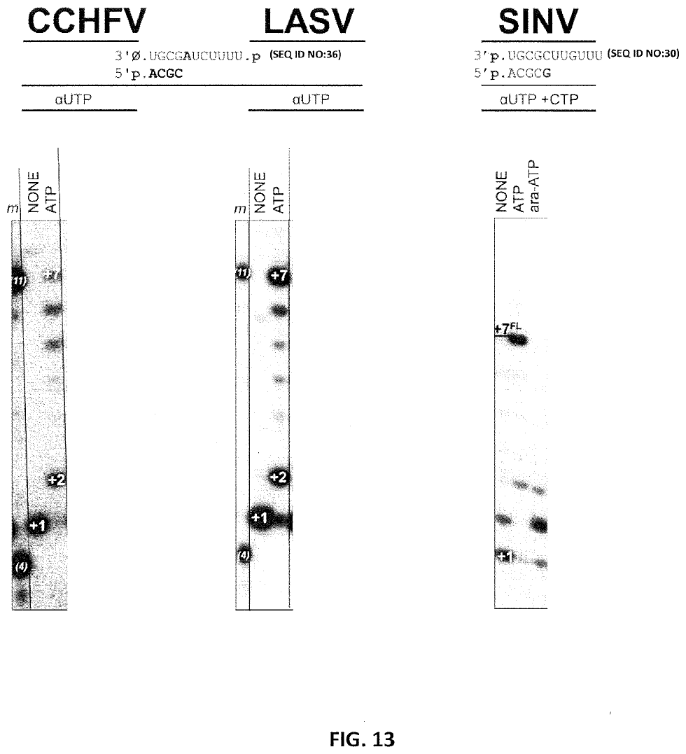

FIG. 13. RNA synthesis activity of recombinant purified CCHFV RdRp, LASV RdRp, and SINV RdRp using the biochemical assay described herein. This data shows that RNA template can be modified such that alpha-³²P-UTP can be used to monitor RdRp activity. SEQ ID NO:36 provides the sequence of the template used in the assay for measuring RdRp activity of recombinant RdRp of CCHFV and LASV. SEQ ID NO:30 provides the sequence of the template used in the assay for measuring RdRp activity of recombinant RdRp of SINV.

### Example 5: Development of a High Throughput Fluorescence-Based Assay for Primed RdRp Activity (HTP-FBA-pRdRa) of a Viral Recombinant Purified RdRp

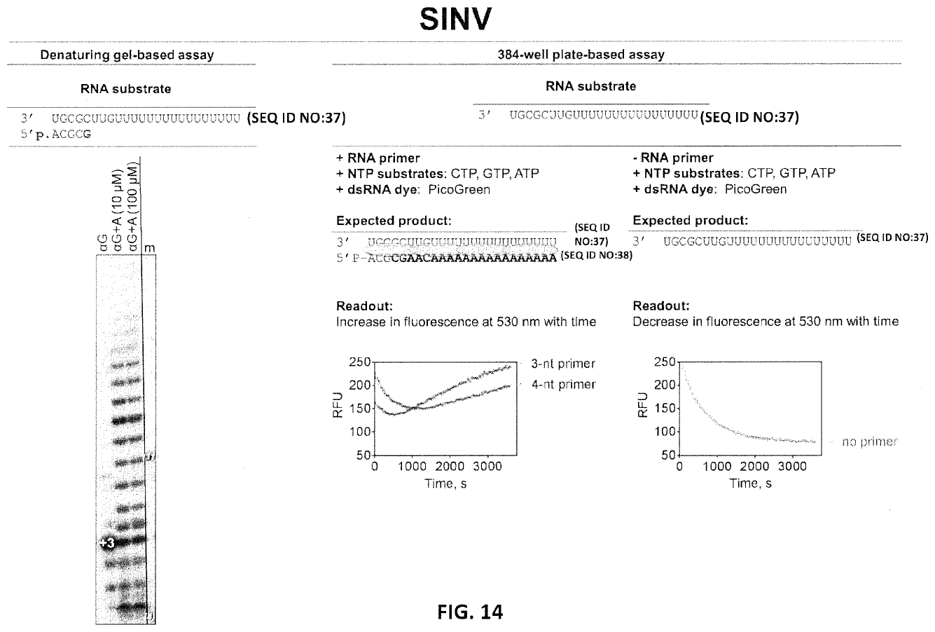

FIG. 14. A high-throughput assay for measuring RdRp activity by utilizing a fluorescent dye that intercalates between dsRNA was developed.

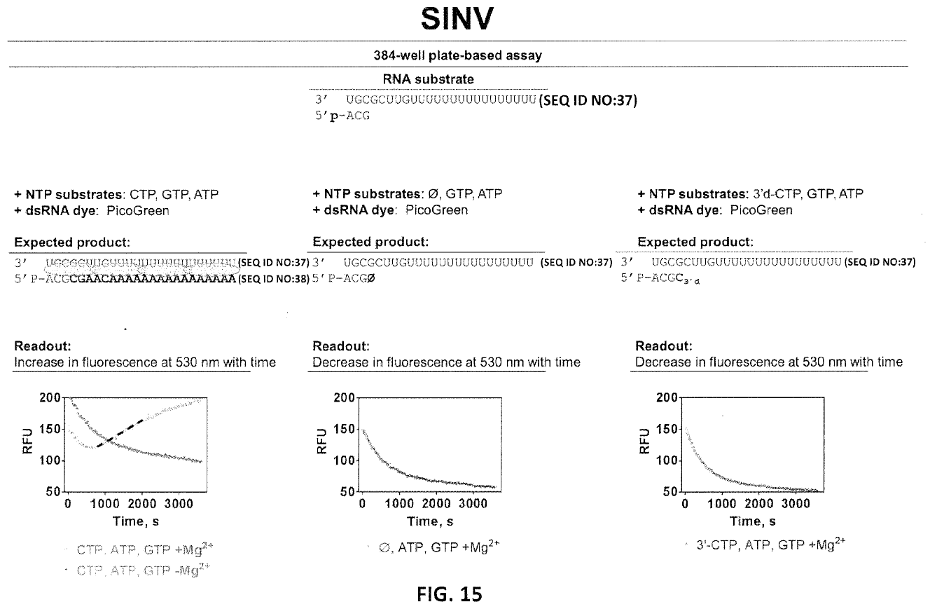

FIG. 15. Nucleotide substrate specificity of a high through-put fluorescence-based assay for primed RdRp activity (HTP-FBA-pRdRa) and inhibtion of thereof by a NTP-analogue. Slope (black dotted line) of the time dependent increase in relative fluorescence units (RFU) reflects the velocity of dsRNA formation as a result of RNA synthesis by SINV RdRp.

SEQ ID NO:37 (5′-UUUUUUUUUUUUUUUUGUUCGCGU-3′) provides the sequence of the template used in the RdRp activity assay. SEQ ID NO: 38 (5′-ACGCGAACAAAAAAAAAAAAAAAA-3′) provides the sequence of the nucleic acid sysnthesized by extension of the primer by RdRp activity.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for assaying activity of a recombinant RNA-dependent RNA polymerase (RdRp) complex of a single-stranded RNA (ss-RNA) virus or a recombinant monomeric RdRp of a ss-RNA virus, the method comprising:
incubating a reaction mixture comprising:
(i) the recombinant RdRp complex or the recombinant monomeric RdRp;
(ii) a RNA substrate comprising:
(a) a primer phosphorylated at the 5′-end and comprising at least three nucleotides, and
(b) a template comprising at least seven nucleotides,
wherein the three nucleotides of the primer are complementary to the 3′-end of the template; and
(iii) nucleotides;
detecting incorporation of at least one nucleotide complementary to the template into the primer,
wherein incorporation of at least one nucleotide into the primer indicates that the recombinant RdRp complex or the recombinant monomeric RdRp is active and copies the template in a base-specific manner.

2. The method of claim 1, wherein the recombinant RdRp complex is from a negative sense ss-RNA virus selected from the group consisting of EBOV, Flu virus, and RSV.

3. The method of claim 1, wherein the recombinant monomeric RdRp is from a negative sense ss-RNA virus selected from the group consisting of SINV Hanta virus, CCHV, and LASV or from a positive sense ss-RNA virus selected from the group consisting of HCV or Zika virus.

4. The method of any of claims 1-3, wherein the reaction mixture comprises a candidate agent, wherein lack of complete extension of the primer indicates that the candidate agent is an inhibitor of the RdRp complex or the monomeric RdRp.

5. The method of any of claims 1-4, wherein the recombinant RdRp is expressed as fusion protein comprising a purification tag.

6. The method of claim 1, wherein the virus is EBOV, the recombinant EBOV RdRp complex comprise an EBOV L polypeptide and an EBOV VP35 polypeptide, wherein incorporation of the at least one nucleotide into the primer indicates that the recombinant EBOV RdRp is active.

7. The method of claim 6, wherein the recombinant EBOV RdRp further comprises an EBOV VP30 polypeptide.

8. The method of any of the preceding claims, wherein the primer is at least four nucleotides long.

9. The method of any of the preceding claims, wherein the template is eleven nucleotides long.

10. The method of any of the preceding claims, wherein the reaction mixture comprises Mg²⁺.

11. The method of any of claims 6-10, wherein the reaction mixture comprises a candidate agent, wherein a lack of incorporation of the at least one nucleotide or an incomplete extension of the primer indicates that the candidate agent is an inhibitor of the EBOV RdRp.

12. A polyprotein comprising an amino acid sequence for Ebola virus (EBOV) L polypeptide fused via a linker to an amino acid sequence for EBOV VP35 polypeptide, wherein the linker is a cleavable linker.

13. The polyprotein of claim 12, further comprising an amino acid for EBOV VP30 polypeptide.

14. The polyprotein of claim 12 or 13, wherein the cleavable linker comprises a sequence cleaved by a protease.

15. The polyprotein of any of claims 12-14, further comprising a tag fused to the polyprotein.

16. The polyprotein of claim 15, wherein the tag comprises a purification tag selected from the group consisting of (Histidine)₆ and streptavidin.

17. A nucleic acid encoding the polyprotein of any one of claims 12-16.

18. The nucleic acid of claim 17, comprising a promoter operably linked to a nucleic acid sequence encoding the polyprotein.

19. A host cell expressing the polyprotein of any one of claims 12-18.

20. A host cell comprising the nucleic acid of 17 or 18.

21. The host cell of claim 19 or 20, wherein the host cell is an insect cell.

22. A system for producing a recombinant Ebola virus (EBOV) RNA-dependent RNA polymerase comprising L polypeptide and VP35 polypeptide, comprising:
a nucleic acid encoding a polyprotein comprising an amino acid sequence of EBOV L polypeptide and an amino acid sequence of EBOV VP35 polypeptide, wherein the amino acid sequence of the L polypeptide is separated from the amino acid sequence for the VP35 polypeptide by the amino acid for a proteolytic cleavage site;
a nucleic acid encoding a protease that cleaves the cleavage site.

23. The system of claim 22, wherein the protease is a tobacco etch virus (TEV) protease.

24. The system of claim 22 or 23, wherein the polyprotein further comprises an amino acid sequence of EBOV VP30 polypeptide.

25. A host cell comprising:
a nucleic acid encoding a polyprotein comprising an amino acid sequence of EBOV L polypeptide and an amino acid sequence of EBOV VP35 polypeptide, wherein the amino acid sequence of the L polypeptide is separated from the amino acid sequence for the VP35 polypeptide by the amino acid sequence for a proteolytic cleavage site;
a nucleic acid encoding a protease that cleaves the cleavage site.

26. The host cell of claim 25, wherein the protease is a tobacco etch virus (TEV) protease.

27. The host cell of claim 25 or 26, wherein the polyprotein further comprises an amino acid sequence of EBOV VP30 polypeptide.

28. The host cell of any one of claim 25-27, wherein the host cell is an insect cell.

29. The host cell of any one of claim 25-28, comprising a vector comprising the nucleic acid encoding the polyprotein.

30. A kit comprising:
(i) a recombinant EBOV RdRp comprising an EBOV L polypeptide and an EBOV VP35 polypeptide;
(ii) a RNA substrate comprising:
(a) a primer phosphorylated at the 5′-end and comprising at least three nucleotides, and
(b) a template comprising at least four nucleotides,
wherein the three nucleotides of primer are complementary to the 3′-end of the template.

31. The kit of claim 30, further comprising:
(iii) nucleotides.

32. The kit of claim 30 or 31, wherein the primer comprises the sequence: 5′-ACGC-3′.

33. The kit of any one of claims 30-32, wherein the template comprises the sequence:
(SEQ ID NO: 39)
5′-UUUGUUCGCGU-3′.

34. The kit of any one of claims 30-33, wherein the nucleotides comprise ara-CTP.

35. The kit of any one of claims 30-34, wherein the EBOV RdRp further comprises EBOV VP35 polypeptide.

36. The kit of any one of claims 30-35, comprising a buffer for maintaining a pH suitable for enzymatic activity of EBOV RdRp.

37. The kit of any one of claims 30-36, comprising a divalent metal ion Mg²⁺.

38. A method of inhibiting activity of a viral RNA dependent RNA polymerase (RdRp), the method comprising:
contacting the viral RdRp with an NTP analog in an amount effective to inhibit activity of the viral RdRp.

39. The method of claim 38, wherein NTP analog is 2′β-hydroxy-cytidine-5′-triphosphate (ara-CTP), or an analog or derivative thereof.

40. The method of claim 38, wherein contacting the viral RdRp comprises administering the ara-CTP or an analog or derivative thereof to a subject having or suspected of having a viral infection.

41. The method of claim 38, wherein the subject has or is suspected of having Ebola virus infection, RSV infection, Influenza virus infection, zika virus infection, Hanta virus infection, CCHV infection, LASV infection, NiV infection, SINV infection, and/or HCV infection.

42. The method of claim 40, wherein the subject has a viral infection and the administering is for a period of time sufficient to treat the infection.

43. The method of claim 40, wherein the subject is a human.


SEQUENCE LISTING (74 sequences)

SEQ ID NO: 1 - protein, 9 aa, Artificial sequence
  synthetic sequence
FNFARLIVY

SEQ ID NO: 2 - protein, 6 aa, Artificial sequence
  synthetic sequence
SRAIWY

SEQ ID NO: 3 - protein, 6 aa, Artificial sequence
  synthetic sequence
SRLIEA

SEQ ID NO: 4 - protein, 6 aa, Artificial sequence
  synthetic sequence
RRAIAT

SEQ ID NO: 5 - protein, 6 aa, Artificial sequence
  synthetic sequence
GRFFSL

SEQ ID NO: 6 - protein, 14 aa, Artificial sequence
  synthetic sequence
  misc_feature (1)..(1): Xaa can be any naturally occurring amino acid
XGFSYDTRHFDSTV

SEQ ID NO: 7 - protein, 14 aa, Artificial sequence
  synthetic sequence
KMYADDTAGWDTRI

SEQ ID NO: 8 - protein, 14 aa, Artificial sequence
  synthetic sequence
  misc_feature (1)..(1): Xaa can be any naturally occurring amino acid
XLFAFDYTGYDASL

SEQ ID NO: 9 - protein, 14 aa, Artificial sequence
  synthetic sequence
MTVTGDNTKWNECL

SEQ ID NO: 10 - protein, 14 aa, Artificial sequence
  synthetic sequence
IANHIDYEKWNNHQ

SEQ ID NO: 11 - protein, 18 aa, Artificial sequence
  synthetic sequence
ASGVLTTSMGNTITCYVK

SEQ ID NO: 12 - protein, 18 aa, Artificial sequence
  synthetic sequence
GSGQVVTYALNTFTNLVV

SEQ ID NO: 13 - protein, 18 aa, Artificial sequence
  synthetic sequence
PSGCSGTSIFNSMINNLI

SEQ ID NO: 14 - protein, 13 aa, Artificial sequence
  synthetic sequence
MFNMLSTVLGVAA

SEQ ID NO: 15 - protein, 13 aa, Artificial sequence
  synthetic sequence
LRQKGWTILNLLV

SEQ ID NO: 16 - protein, 5 aa, Artificial sequence
  synthetic sequence
CGDDL

SEQ ID NO: 17 - protein, 5 aa, Artificial sequence
  synthetic sequence
SGDDC

SEQ ID NO: 18 - protein, 5 aa, Artificial sequence
  synthetic sequence
YGDDV

SEQ ID NO: 19 - protein, 5 aa, Artificial sequence
  synthetic sequence
SSDDF

SEQ ID NO: 20 - protein, 5 aa, Artificial sequence
  synthetic sequence
QGDNQ

SEQ ID NO: 21 - protein, 6 aa, Artificial sequence
  synthetic sequence
GRMFAM

SEQ ID NO: 22 - protein, 6 aa, Artificial sequence
  synthetic sequence
GRTFGK

SEQ ID NO: 23 - protein, 14 aa, Artificial sequence
  synthetic sequence
CSIITDLSKFNQAF

SEQ ID NO: 24 - protein, 14 aa, Artificial sequence
  synthetic sequence
SSFVTDLEKYNLAF

SEQ ID NO: 25 - protein, 18 aa, Artificial sequence
  synthetic sequence
IEGWCQKLWTIEAISLLD

SEQ ID NO: 26 - protein, 18 aa, Artificial sequence
  synthetic sequence
IEGLQQKLWTSISCAQIS

SEQ ID NO: 27 - protein, 5 aa, Artificial sequence
  synthetic sequence
NGDNQ

SEQ ID NO: 28 - protein, 5 aa, Artificial sequence
  synthetic sequence
MGDNQ

SEQ ID NO: 29 - DNA, 11 bp, Artificial sequence
  synthetic sequence
tgcgcttgttt

SEQ ID NO: 30 - RNA, 11 nt, Artificial sequence
  synthetic sequence
uuuguucgcgu

SEQ ID NO: 31 - RNA, 11 nt, Artificial sequence
  synthetic sequence
acgcgaacaaa

SEQ ID NO: 32 - RNA, 11 nt, Artificial sequence
  synthetic sequence
ugcgcuuuuuu

SEQ ID NO: 33 - RNA, 18 nt, Artificial sequence
  synthetic sequence
ugcgcuuuuuuacccccc

SEQ ID NO: 34 - RNA, 14 nt, Artificial sequence
  synthetic sequence
ugcgcuuguuuauu

SEQ ID NO: 35 - RNA, 15 nt, Artificial sequence
  synthetic sequence
ugcgcuaaguuuauu

SEQ ID NO: 36 - RNA, 11 nt, Artificial sequence
  synthetic sequence
uuuucuagcgu

SEQ ID NO: 37 - RNA, 24 nt, Artificial sequence
  synthetic sequence
uuuuuuuuuuuuuuuuguucgcgu

SEQ ID NO: 38 - RNA, 24 nt, Artificial sequence
  synthetic sequence
acgcgaacaaaaaaaaaaaaaaaa

SEQ ID NO: 39 - RNA, 11 nt, Artificial sequence
  synthetic sequence
uuuguucgcgu

SEQ ID NO: 40 - protein, 5 aa, Artificial sequence
  synthetic sequence
  MISC_FEATURE (1)..(5): The sequence from positions 1 to 5 may be repeated n times.
GSGGS

SEQ ID NO: 41 - protein, 4 aa, Artificial sequence
  synthetic sequence
  MISC_FEATURE (1)..(4): The sequence from positions 1 to 4 may be repeated n times.
GGGS

SEQ ID NO: 42 - protein, 4 aa, Artificial sequence
  synthetic sequence
  MISC_FEATURE (2)..(3): The amino acids at positions 2 and 3 may be any amino acid.
  MISC_FEATURE (4)..(4): The amino acid at position 4 is a hydrophobic amino acid.
PXXX

SEQ ID NO: 43 - protein, 5 aa, Artificial sequence
  synthetic sequence
  MISC_FEATURE (2)..(3): The amino acids at positions 2 and 3 may be any amino acid.
  MISC_FEATURE (4)..(4): The amino acids at position 4 may be a hydrophobic residue.
  MISC_FEATURE (5)..(5): The amino acids at position 5 may be serine or threonine.
PXXXX

SEQ ID NO: 44 - protein, 6 aa, Artificial sequence
  synthetic sequence
  MISC_FEATURE (2)..(2): The amino acid at position 2 may be leucine or glutamine.
PXGMTS

SEQ ID NO: 45 - protein, 5 aa, Artificial sequence
  synthetic sequence
  MISC_FEATURE (2)..(2): The amino acid at position 2 may be leucine or glutamine.
PXGMT

SEQ ID NO: 46 - protein, 7 aa, Artificial sequence
  synthetic sequence
ENLYTQS

SEQ ID NO: 47 - protein, 5 aa, Artificial sequence
  synthetic sequence
DDDDK

SEQ ID NO: 48 - protein, 8 aa, Artificial sequence
  synthetic sequence
LEVLFQGP

SEQ ID NO: 49 - protein, 10 aa, Artificial sequence
  synthetic sequence
CGLVPAGSGP

SEQ ID NO: 50 - protein, 12 aa, Artificial sequence
  synthetic sequence
SLLKSRMVPNFN

SEQ ID NO: 51 - protein, 12 aa, Artificial sequence
  synthetic sequence
SLLIARRMPNFN

SEQ ID NO: 52 - protein, 12 aa, Artificial sequence
  synthetic sequence
SKLVQASASGVN

SEQ ID NO: 53 - protein, 12 aa, Artificial sequence
  synthetic sequence
SSYLKASDAPDN

SEQ ID NO: 54 - protein, 12 aa, Artificial sequence
  synthetic sequence
RPKPQQFFGLMN

SEQ ID NO: 55 - protein, 12 aa, Artificial sequence
  synthetic sequence
SLRPLALWRSFN

SEQ ID NO: 56 - protein, 12 aa, Artificial sequence
  synthetic sequence
SPQGIAGQRNFN

SEQ ID NO: 57 - protein, 14 aa, Artificial sequence
  synthetic sequence
DVDERDVRGFASFL

SEQ ID NO: 58 - protein, 12 aa, Artificial sequence
  synthetic sequence
SLPLGLWAPNFN

SEQ ID NO: 59 - protein, 12 aa, Artificial sequence
  synthetic sequence
SLLIFRSWANFN

SEQ ID NO: 60 - protein, 12 aa, Artificial sequence
  synthetic sequence
SGVVIATVIVIT

SEQ ID NO: 61 - protein, 12 aa, Artificial sequence
  synthetic sequence
SLGPQGIWGQFN

SEQ ID NO: 62 - protein, 12 aa, Artificial sequence
  synthetic sequence
KKSPGRVVGGSV

SEQ ID NO: 63 - protein, 12 aa, Artificial sequence
  synthetic sequence
PQGLLGAPGILG

SEQ ID NO: 64 - protein, 31 aa, Artificial sequence
  synthetic sequence
HGPEGLRVGFYESDVMGRGHARLVHVEEPHT

SEQ ID NO: 65 - protein, 12 aa, Artificial sequence
  synthetic sequence
GPQGLAGQRGIV

SEQ ID NO: 66 - protein, 12 aa, Artificial sequence
  synthetic sequence
GGSGQRGRKALE

SEQ ID NO: 67 - protein, 12 aa, Artificial sequence
  synthetic sequence
SLSALLSSDIFN

SEQ ID NO: 68 - protein, 12 aa, Artificial sequence
  synthetic sequence
SLPRFKIIGGFN

SEQ ID NO: 69 - protein, 12 aa, Artificial sequence
  synthetic sequence
SLLGIAVPGNFN

SEQ ID NO: 70 - protein, 12 aa, Artificial sequence
  synthetic sequence
FFKNIVTPRTPP

SEQ ID NO: 71 - DNA, 4 bp, Artificial sequence
  synthetic sequence
acgc

SEQ ID NO: 72 - RNA, 11 nt, Artificial sequence
  synthetic sequence
ugcgcuuuuuu

SEQ ID NO: 73 - RNA, 11 nt, Artificial sequence
  synthetic sequence
ugcgaucuuuu

SEQ ID NO: 74 - RNA, 18 nt, Artificial sequence
  synthetic sequence
ugcgcuuuuuuacccccc